(19) United States
(12) Patent Application Publication
(10) Pub. No.: US 2022/0193439 A1
(43) Pub. Date: Jun. 23, 2022

(54) TREATMENTS FOR EYE INFECTION

(71) Applicants: Desmond Christopher Adler, Bedford, MA (US); Jason Hill, Auburndale, MA (US); David Usher, Waltham, MA (US); Mikhail Smirnov, North Andover, MA (US); Ahalya Viswanathan, Arlington, MA (US)

(72) Inventors: Desmond Christopher Adler, Bedford, MA (US); Jason Hill, Auburndale, MA (US); David Usher, Waltham, MA (US); Mikhail Smirnov, North Andover, MA (US); Ahalya Viswanathan, Arlington, MA (US)

(21) Appl. No.: 17/554,651

(22) Filed: Dec. 17, 2021

Related U.S. Application Data
(60) Provisional application No. 63/126,648, filed on Dec. 17, 2020.
(60) Provisional application No. 63/239,155, filed on Aug. 31, 2021.

(51) Int. Cl. A61N 5/06; A61F 9/00; A61K 41/00; A61P 31/04

(52) U.S. Cl. CPC A61N 5/062; A61F 9/0008; A61N 5/0624; A61N 2005/0661; A61P 31/04; A61N 2005/0626; A61K 41/008

(57) ABSTRACT

An example antimicrobial treatment system includes an illumination system configured to deliver illumination that activates a photosensitizing agent applied to a cornea. The system also includes a controller configured to control the illumination system. The controller detects an ulcerative region on a cornea and causes the illumination system to deliver the illumination to activate the photosensitizing agent applied to the ulcerative region according to a set of parameters for treating the ulcerative region. The illumination is restricted to the ulcerative region, and activation of the photosensitizing agent in the ulcerative region generates an antimicrobial effect.

### CROSS-REFERENCE TO RELATED APPLICATIONS

The present application claims priority to, and benefit of, U.S. Provisional Patent Application Ser. No. 63/126,648, filed on Dec. 17, 2020, and U.S. Provisional Patent Application Ser. No. 63/239,155, filed on Aug. 31, 2021, the contents of these applications being incorporated entirely herein by reference.

### BACKGROUND

### Field

The present disclosure relates to formulations, systems, and methods for treating an eye, and more particularly, to formulations, systems, and methods for antimicrobial and/or anti-parasitic treatment of infections associated, for example, with ulcerative keratitis or blepharitis.

### Description of Related Art

Various eye conditions are characterized by infection, inflammation, and other dysfunction caused by bacteria and other parasites. Antimicrobial and/or anti-parasitic pharmaceuticals are often employed to treat such conditions.

For example, bacterial keratitis is an infection of the cornea caused by bacteria, such as Staphylococcus aureus and Pseudomonas aeruginosa. Amoebic keratitis is an infection of the cornea caused by amoeba, such as Acanthamoeba. Fungal keratitis is an infection of the cornea caused by fungi. Such eye infections may cause pain, reduced vision, light sensitivity, and tearing or discharge. Superficial keratitis is an infection that is generally limited to the uppermost layers of the cornea, and after healing, usually leaves no scar on the cornea. On the other hand, ulcerative keratitis is characterized by an infection that has progressed to a state of epithelial disruption, stromal penetration, and ulcer formation. If left untreated, ulcerative keratitis can cause blindness. Conventional antimicrobial pharmaceuticals are often less effective at treating ulcerative keratitis. As such, treatment of ulcerative keratitis may eventually require a corneal transplant, which is associated with high cost and morbidity rate.

For another example, blepharitis is a chronic eye condition characterized by inflammation of the eyelid, often concurrent with Meibomian gland dysfunction and severe dryness of the ocular surface. The Meibomian glands are glands that line the margin of the eyelids and secrete oil which coats the surface of an eye and keeps the water component of tears from evaporating. Blepharitis is thought in many cases to be caused by over-proliferation of demodex mites residing in the eyelash follicles and the Meibomian glands, along with infection by Bacillus oleronius which spreads with the mites. Current blepharitis treatments include systemic or topical application of pharmaceuticals with anti-parasitic and/or antimicrobial effects, which may be effective but may also induce systemic or local toxicity and require chronic administration.

### SUMMARY

Formulations, systems, and methods provide antimicrobial and/or anti-parasitic treatment of infections associated, for example, with ulcerative keratitis. For example, a method for antimicrobial treatment includes detecting an ulcerative region on a cornea, applying a photosensitizing agent to the cornea, and delivering, with an illumination system, an illumination that activates the photosensitizing agent applied to the ulcerative region according to a set of parameters for treating the ulcerative region. The illumination is restricted to the ulcerative region, and activation of the photosensitizing agent in the ulcerative region generates an antimicrobial effect.

For example, an antimicrobial treatment system includes an illumination system configured to deliver illumination that activates a photosensitizing agent applied to a cornea. The system also includes a controller configured to control the illumination system. The controller includes one or more processors and one or more computer readable media. The one or more processors are configured to execute instructions from the computer readable media to cause the controller to detect an ulcerative region on a cornea and cause the illumination system to deliver the illumination to activate the photosensitizing agent applied to the ulcerative region according to a set of parameters for treating the ulcerative region. The illumination is restricted to the ulcerative region, and activation of the photosensitizing agent in the ulcerative region generates an antimicrobial effect.

### BRIEF DESCRIPTION OF THE DRAWINGS

FIG. 1 illustrates an example treatment system including a drug formulation with a photosensitizing agent and an illumination system configured to deliver illumination that activates the photosensitizing agent to produce antimicrobial effects.

FIG. 2 illustrates a cornea with an ulcerative region, where ultraviolet (UV) light is applied as a broad, homogeneous beam to a treatment zone extending across approximately the entire cornea.

FIG. 3 illustrates a cornea with an ulcerative region, where UV light is applied to multiple treatment zones according to different parameters to produce different treatment effects, respectively.

FIG. 4 illustrates an example treatment system including a drug formulation with a photosensitizing agent, an illumination system configured to deliver illumination that activates the photosensitizing agent to produce antimicrobial effects, and a controller with a user interface and imaging system that can be used to detect an ulcerative region on a cornea.

FIG. 5A illustrates an example image of a cornea with an ulcerative region shown on a display of a user interface, where the user interface is employed to identify a point in the ulcerative region.

FIG. 5B illustrates an example image of a cornea with an ulcerative region shown on a display of a user interface, where the user interface is employed to produce a tracing that identifies a border of the ulcerative region.

FIG. 6A illustrates mean concentration of oxygen (O₂) in a cornea as a function of time when the cornea is exposed to a concentration of 21% ambient O₂ during a corneal treatment that activates a riboflavin concentration of 0.125% with UV light having irradiances of 2.5 mW/cm², 10 mW/cm², 40 mW/cm², and 160 mW/cm².

FIG. 6B illustrates mean concentration of O₂ in a cornea as a function of time when the cornea is exposed to a concentration of 21% ambient O₂ during a corneal treatment that activates a riboflavin concentration of 0.25% with UV light having irradiances of 2.5 mW/cm², 10 mW/cm², 40 mW/cm², and 160 mW/cm².

FIG. 6C illustrates mean concentration of O₂ in a cornea as a function of time when the cornea is exposed to a concentration of 21% ambient O₂ during a corneal treatment that activates a riboflavin concentration of 0.5% with UV light having irradiances of 2.5 mW/cm², 10 mW/cm², 40 mW/cm², and 160 mW/cm².

FIG. 6D illustrates mean concentration of O₂ in a cornea as a function of time when the cornea is exposed to a concentration of 21% ambient O₂ during a corneal treatment that activates a riboflavin concentration of 1% with UV light having irradiances of 2.5 mW/cm², 10 mW/cm², 40 mW/cm², and 160 mW/cm².

FIG. 7A illustrates mean concentration of O₂ in a cornea as a function of time when the cornea is exposed to a concentration of 90% ambient O₂ during a corneal treatment that activates a riboflavin concentration of 0.125% with UV light having irradiances of 2.5 mW/cm², 10 mW/cm², 40 mW/cm², and 160 mW/cm².

FIG. 7B illustrates mean concentration of O₂ in a cornea as a function of time when the cornea is exposed to a concentration of 90% ambient O₂ during a corneal treatment that activates a riboflavin concentration of 0.25% with UV light having irradiances of 2.5 mW/cm², 10 mW/cm², 40 mW/cm², and 160 mW/cm².

FIG. 7C illustrates mean concentration of O₂ in a cornea as a function of time when the cornea is exposed to a concentration of 90% ambient O₂ during a corneal treatment that activates a riboflavin concentration of 0.5% with UV light having irradiances of 2.5 mW/cm², 10 mW/cm², 40 mW/cm², and 160 mW/cm².

FIG. 7D illustrates mean concentration of O₂ in a cornea as a function of time when the cornea is exposed to a concentration of 90% ambient O₂ during a corneal treatment that activates a riboflavin concentration of 1% with UV light having irradiances of 2.5 mW/cm², 10 mW/cm², 40 mW/cm², and 160 mW/cm².

FIG. 8A illustrates mean concentration of singlet oxygen (O₁) in a cornea as a function of time when the cornea is exposed to a concentration of 21% ambient O₂ during a corneal treatment that activates a riboflavin concentration of 0.125% with UV light having irradiances of 2.5 mW/cm², 10 mW/cm², 40 mW/cm², and 160 mW/cm².

FIG. 8B illustrates mean concentration of O₁ in a cornea as a function of time when the cornea is exposed to a concentration of 21% ambient O₂ during a corneal treatment that activates a riboflavin concentration of 0.25% with UV light having irradiances of 2.5 mW/cm², 10 mW/cm², 40 mW/cm², and 160 mW/cm².

FIG. 8C illustrates mean concentration of O₁ in a cornea as a function of time when the cornea is exposed to a concentration of 21% ambient O₂ during a corneal treatment that activates a riboflavin concentration of 0.5% with UV light having irradiances of 2.5 mW/cm², 10 mW/cm², 40 mW/cm², and 160 mW/cm².

FIG. 8D illustrates mean concentration of O₁ in a cornea as a function of time when the cornea is exposed to a concentration of 21% ambient O₂ during a corneal treatment that activates a riboflavin concentration of 1% with UV light having irradiances of 2.5 mW/cm², 10 mW/cm², 40 mW/cm², and 160 mW/cm².

FIG. 9A illustrates mean concentration of O₁ in a cornea as a function of time when the cornea is exposed to a concentration of 90% ambient O₂ during a corneal treatment that activates a riboflavin concentration of 0.125% with UV light having irradiances of 2.5 mW/cm², 10 mW/cm², 40 mW/cm², and 160 mW/cm².

FIG. 9B illustrates mean concentration of O₁ in a cornea as a function of time when the cornea is exposed to a concentration of 90% ambient O₂ during a corneal treatment that activates a riboflavin concentration of 0.25% with UV light having irradiances of 2.5 mW/cm², 10 mW/cm², 40 mW/cm², and 160 mW/cm².

FIG. 9C illustrates mean concentration of O₁ in a cornea as a function of time when the cornea is exposed to a concentration of 90% ambient O₂ during a corneal treatment that activates a riboflavin concentration of 0.5% with UV light having irradiances of 2.5 mW/cm², 10 mW/cm², 40 mW/cm², and 160 mW/cm².

FIG. 9D illustrates mean concentration of O₁ in a cornea as a function of time when the cornea is exposed to a concentration of 90% ambient O₂ during a corneal treatment that activates a riboflavin concentration of 1% with UV light having irradiances of 2.5 mW/cm², 10 mW/cm², 40 mW/cm², and 160 mW/cm².

FIG. 10A illustrates mean concentration of hydrogen peroxide (H₂O₂) in a cornea as a function of time when the cornea is exposed to a concentration of 21% ambient O₂ during a corneal treatment that activates a riboflavin concentration of 0.125% with UV light having irradiances of 2.5 mW/cm², 10 mW/cm², 40 mW/cm², and 160 mW/cm².

FIG. 10B illustrates mean concentration of H₂O₂ in a cornea as a function of time when the cornea is exposed to a concentration of 21% ambient O₂ during a corneal treatment that activates a riboflavin concentration of 0.25% with UV light having irradiances of 2.5 mW/cm², 10 mW/cm², 40 mW/cm², and 160 mW/cm².

FIG. 10C illustrates mean concentration of H₂O₂ in a cornea as a function of time when the cornea is exposed to a concentration of 21% ambient O₂ during a corneal treatment that activates a riboflavin concentration of 0.5% with UV light having irradiances of 2.5 mW/cm², 10 mW/cm², 40 mW/cm², and 160 mW/cm².

FIG. 10D illustrates mean concentration of H₂O₂ in a cornea as a function of time when the cornea is exposed to a concentration of 21% ambient O₂ during a corneal treatment that activates a riboflavin concentration of 1% with UV light having irradiances of 2.5 mW/cm², 10 mW/cm², 40 mW/cm², and 160 mW/cm².

FIG. 11A illustrates mean concentration of H₂O₂ in a cornea as a function of time when the cornea is exposed to a concentration of 90% ambient O₂ during a corneal treatment that activates a riboflavin concentration of 0.125% with UV light having irradiances of 2.5 mW/cm², 10 mW/cm², 40 mW/cm², and 160 mW/cm².

FIG. 11B illustrates mean concentration of H₂O₂ in a cornea as a function of time when the cornea is exposed to a concentration of 90% ambient O₂ during a corneal treatment that activates a riboflavin concentration of 0.25% with UV light having irradiances of 2.5 mW/cm², 10 mW/cm², 40 mW/cm², and 160 mW/cm².

FIG. 11C illustrates mean concentration of H₂O₂ in a cornea as a function of time when the cornea is exposed to a concentration of 90% ambient O₂ during a corneal treatment that activates a riboflavin concentration of 0.5% with UV light having irradiances of 2.5 mW/cm², 10 mW/cm², 40 mW/cm², and 160 mW/cm².

FIG. 11D illustrates mean concentration of H₂O₂ in a cornea as a function of time when the cornea is exposed to a concentration of 90% ambient O₂ during a corneal treatment that activates a riboflavin concentration of 1% with UV light having irradiances of 2.5 mW/cm², 10 mW/cm², 40 mW/cm², and 160 mW/cm².

FIG. 12A illustrates a graph of predicted production of H₂O₂ and reactive oxygen species (ROS) (i.e., superoxide, singlet oxygen, and hydroxyl radical (OH)) for example protocols A-D.

FIG. 12B illustrates a graph of bacterial death rates for the example protocols A-D of FIG. 12A.

FIG. 13 illustrates an example system for an in vitro study of keratitis treatments.

FIG. 14A illustrates an example sample for an in vitro study of keratitis treatments.

FIG. 14B illustrates example delivery of the laser beam to selected quadrants of the sample of FIG. 14A for the study.

FIG. 14C illustrates results after the quadrants of the sample of FIG. 14B have been exposed to different conditions for the study.

FIG. 15 illustrates an eye, an upper eyelid, a lower eyelid, Meibomian glands on the respective eyelids, and eyelashes extending from the respective eyelids with corresponding follicles.

FIG. 16A illustrates an example system for applying photodynamic therapy (PDT) as a treatment for blepharitis.

FIG. 16B illustrates an example application of PDT as a treatment for blepharitis.

FIG. 16C illustrates an example image where Meibomian glands can be detected for treatment for blepharitis.

FIG. 17 illustrates an example application of cryotherapy as a treatment for blepharitis.

FIG. 18 illustrates an example application of radiofrequency (RF) therapy as a treatment for blepharitis.

FIG. 19 illustrates an example application of direct thermal therapy as a treatment for blepharitis.

FIG. 20 illustrates an example application of direct thermal therapy via laser irradiation as a treatment for blepharitis.

FIG. 21 illustrates an example application of ultrasound therapy as a treatment for blepharitis.

FIG. 22A illustrates an example procedure for the studying effects of riboflavin/UVA laser treatments under normoxic conditions on Pseudomonas aeruginosa.

FIG. 22B illustrates an example procedure for the studying effects of riboflavin/UVA laser treatments under hyperoxic conditions on Pseudomonas aeruginosa.

FIG. 22C illustrates an example procedure for the studying effects of riboflavin/UVA light emitting diode (LED) treatments under normoxic conditions on Pseudomonas aeruginosa.

FIG. 23 illustrates a graph showing effect of 0.1% riboflavin/UVA laser treatments on cell death of Pseudomonas aeruginosa under normoxic conditions.

FIG. 24 illustrates a graph showing effect of 0.5% riboflavin/UVA laser treatments on cell death of Pseudomonas aeruginosa under normoxic conditions.

FIG. 25 illustrates a graph showing effect of 0.1% riboflavin/UVA laser treatments on cell death of Pseudomonas aeruginosa under hyperoxic conditions.

FIG. 26 illustrates a graph showing effect of 0.22% riboflavin/UVA laser treatments on cell death of Pseudomonas aeruginosa under hyperoxic conditions.

FIG. 27 illustrates a graph showing effect of 0.2% riboflavin/UVA laser treatments on cell death of Pseudomonas aeruginosa under hyperoxic conditions.

FIG. 28 illustrates a graph showing effect of 0.1% riboflavin/UVA LED treatments on cell death of Pseudomonas aeruginosa under normoxic conditions.

FIG. 29 illustrates graphs comparing average cell death for Pseudomonas aeruginosa and production of H₂O₂ (μM) from riboflavin/UVA laser treatments.

FIG. 30 illustrates a graph comparing production of H₂O₂ (μM) vs. riboflavin concentration in riboflavin/UVA laser treatments.

### DESCRIPTION

Cytotoxic chemical species, such as reactive oxygen and hydrogen peroxide, are generated when a photosensitizing agent, such as riboflavin, is applied to the cornea and exposed to activating illumination, such as ultraviolet (UV) light. Because these chemical species are highly toxic to bacteria, fungus, and/or amoeba, infection associated with ulcerative keratitis can be treated by activating a photosensitizing agent applied to the cornea. Such treatment is known as photodynamic therapy (PDT). By achieving effective treatment of active infection of the cornea with the photosensitizing agent, the need for a corneal transplant is significantly reduced.

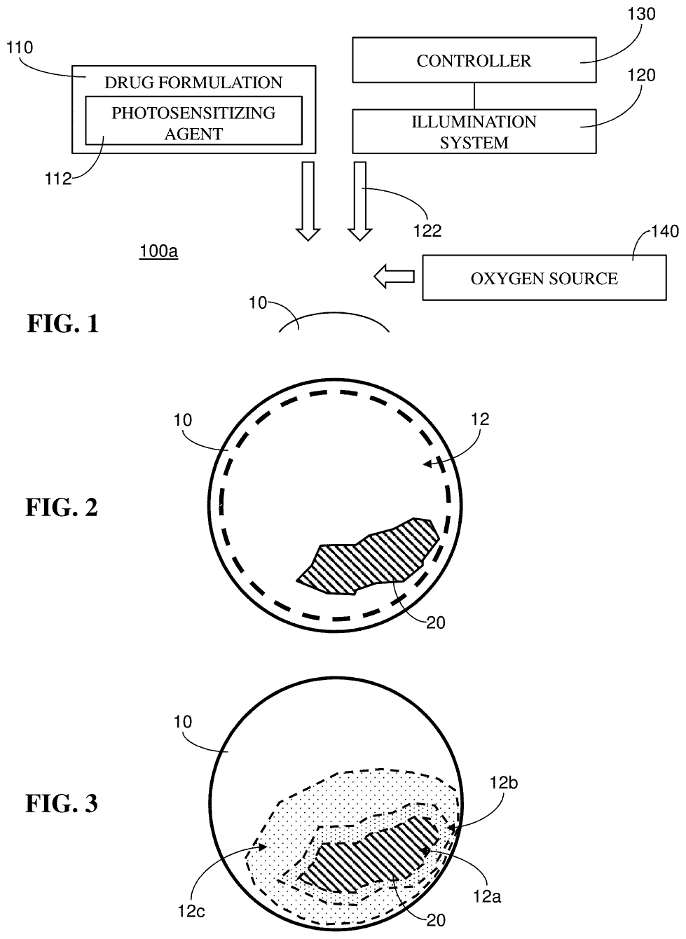

According to an implementation of an example treatment system 100a shown in FIG. 1, a drug formulation 110 including a photosensitizing agent 112 is applied to a cornea 10 (e.g., as eye drops) and an illumination system 120 is operated to deliver illumination (radiation) 122 that activates the photosensitizing agent 112. The illumination system 120 may employ a light emitting diode (LED) or a laser source to deliver the illumination 122. The treatment system 100a also may also include a controller 130 to control certain treatment parameters. For instance, the controller 130 can be coupled to the illumination system 120 to control parameters relating to the illumination 122, such as instantaneous power, average irradiance, total dose, and pulsing characteristics, any and all of which can be varied spatially according to the location and size of an infection. Additionally, oxygen from an oxygen source 140 may be delivered to the cornea 10 to determine a level of ambient oxygen for the treatment, the effect of which is described further herein.

Aspects of the present disclosure provide drug formulations and/or illumination systems that are specifically optimized to sterilize infected regions of corneal tissue for an effective antimicrobial treatment. For instance, example drug formulations can be optimized to reduce the required amount of time that the cornea is exposed to the photosensitizing agent (also known as soak time) before activating illumination is delivered to the cornea. Example drug formulations can be optimized to maximize the antimicrobial effect inside an ulcerative region to reduce the infectious burden associated with the keratitis. Because the activation of the photosensitizing agent may also result in corneal cross-linking, example illumination systems can be optimized to generate cross-linking activity outside the ulcerative region to increase resistance to enzymatic digestion and growth of the ulcerative region. Example illumination systems can be optimized to apply illumination to custom treatment zones that correspond to the size and location of the ulcerative region specific to each patient.

To date, treatments involving the activation of photosensitizing agents are more suitable for generating cross-linking activity to treat ectactic disorders, such as keratoconus. Such cross-linking treatments generally apply drug formulations with concentrations of riboflavin of approximately 0.1% to 0.2% to the cornea with approximately ten to thirty minutes of soak time. Additionally, such treatments activate the drug formulations with illumination systems that apply broad, homogenous beams of UV light with a diameter of approximately 9 mm diameter with illumination parameters optimized for generating cross-linking activity rather than antimicrobial effects. If applied to treat ulcerative keratitis, this broad homogenous beam would expose approximately the entire corneal surface to the same amount of non-optimized illumination regardless of the size or location of the ulcerative region.

For instance, FIG. 2 illustrates a cornea 10 with an ulcerative region 20. According to the approach shown in FIG. 2, the illumination system 120 delivers the illumination 122 to the cornea 10 as a broad, homogeneous beam to expose a single treatment zone 12 (indicated by the dashed line) which extends approximately across the entire cornea 10. The ulcerative region 20, however, is smaller than the treatment zone 12. As such, all portions of the treatment zone 12 inside and outside the ulcerative region 20 are exposed to the same illumination, even though different treatment effects may be desired inside and outside the ulcerative region 20.

FIG. 3 also illustrates the cornea 10 with the ulcerative region 20. In contrast to the approach shown in FIG. 2, the approach shown in FIG. 3 produces different treatment effects in multiple treatment zones 12a-c extending across the cornea 10. The first treatment zone 12a corresponds to the region inside the ulcerative region 20. The second treatment zone 12b corresponds to the border of the ulcerative region 20. The third treatment zone 12c corresponds to a peripheral region beyond the border of the ulcerative region 20. The parameters for the illumination system 120, such as instantaneous power, average irradiance, total dose, and pulsing characteristics, can be selected via the controller 130 to generate different treatment effects for the treatment zones 12a-c. Using a digital micromirror device (DMD), laser scanning, or other precise imaging technique, for instance, the illumination system 120 can accurately apply illumination to a specific treatment zone (in contrast to a broad application extending across approximately the entire cornea). For instance, the illumination system 120 can deliver the illumination 122 to the first treatment zone 12a according to parameters optimized for maximum antimicrobial effect inside the ulcerative region 20. On the other hand, the illumination system 120 can deliver the illumination 122 to the second treatment zone 12b according to parameters optimized to generate strong cross-linking activity along the border of the ulcerative region 20 to reduce enzymatic digestion and resist the growth of the ulcerative region 20. Meanwhile, the illumination system 120 can deliver the illumination 122 to the third treatment zone 12c according to parameters optimized to generate more modest cross-linking activity to stabilize the cornea 10.

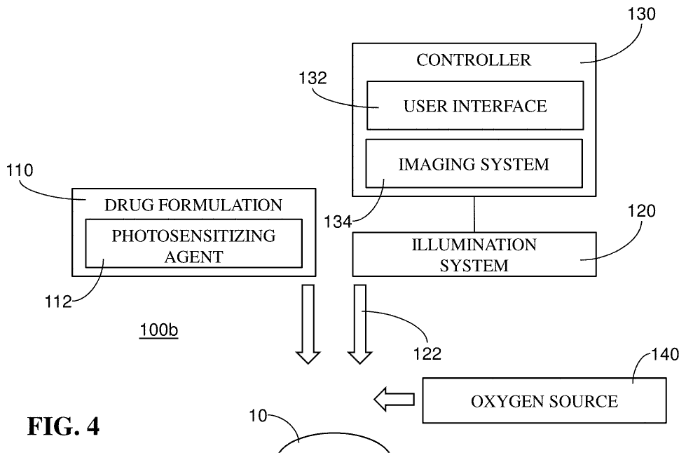

FIG. 4 illustrates another example treatment system 100b. Similar to the example treatment system 100a shown in FIG. 1, an implementation of the example treatment system 100b involves applying the drug formulation 110 including the photosensitizing agent 112 to the cornea 10 (e.g., as eye drops) and operating the illumination system 120 to deliver illumination 122 that activates the photosensitizing agent 112. Additionally, oxygen from an oxygen source 140 may be delivered to the cornea 10 to determine a level of ambient oxygen for the treatment.

Like the treatment system 100a, the treatment system 100b also includes a controller 130 to control certain treatment parameters. As shown in FIG. 4, the controller 130 also includes a user interface 132 and an imaging system 134. The user interface 132 can receive input from an operator to control the treatment parameters implemented by the controller 130. Such input can be received, for instance, via a keyboard, computer mouse, touchscreen, stylus, dials, buttons, or the like. The user interface 132 can also provide the operator with treatment information. Such treatment information can be provided, for instance, visually and/or audibly via a display, illuminated indicators, speakers, or the like.

Additionally, the controller 130 includes an imaging system 134 with a camera that can provide images of the cornea 10 to the operator via a display of the user interface 132. In particular, the operator can use information from the images from the imaging system 134 to control the treatment parameters via the user interface 132. For instance, the images can show the location, size, and shape of the ulcerative region 20 on the cornea 10. The images may be acquired immediately prior to treatment to ensure accurate identification of the ulcerative region 20.

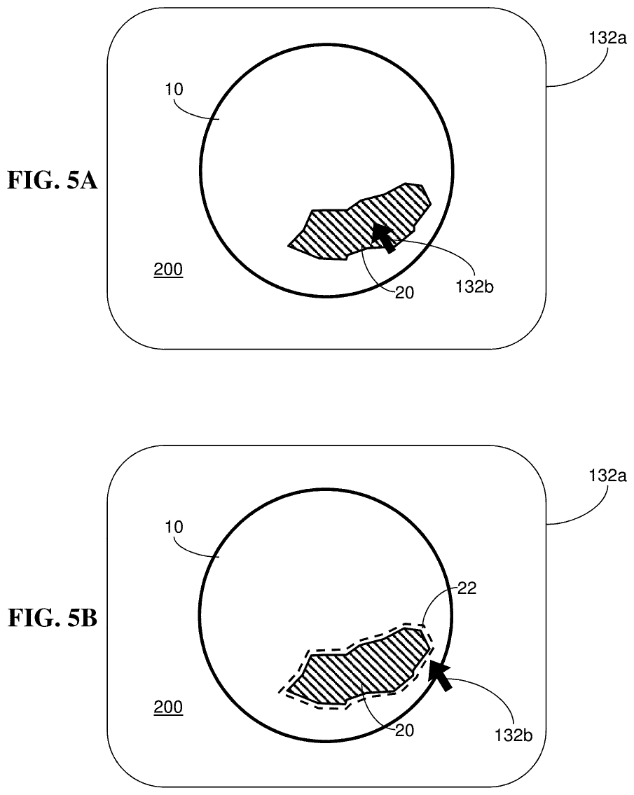

FIG. 5A illustrates an example image 200 from the imaging system 134 as shown on a display 132a of the user interface 132. The image 200 shows the cornea 10 with the ulcerative region 20. The user interface 132 also provides a cursor 132b that is also shown on the display 132a and can be moved over the image 200 by the operator, for instance, by manipulation of a computer mouse. According to the example approach illustrated in FIG. 5A, the operator can move the cursor 132b over a point in the ulcerative region 20 shown in the image 200 and correspondingly click a button on a computer mouse to register the point in the ulcerative region 20 with the controller 130. (Alternative user interface tools are also contemplated; for instance, the user interface 132 may provide a touchscreen that the operator can tap to identify a point in the ulcerative region 20 in the image 200.)

Once the operator identifies a point in the ulcerative region 20 in the image 200 via the user interface 132, the controller 130 can process the image 200 to further identify the border of the ulcerative region 20, for instance, via edge detection techniques. Due to the heterogeneous visual appearance of the ulcerative region 20, the process of identifying the ulcerative region 20 within the image 200 is simplified and made more robust by having the operator identify an initial point inside the ulcerative region 20 from which the controller 130 can subsequently search for the border.

FIG. 5B also illustrates the example image 200 as shown on the display 132a. Similar to FIG. 5A, the image 200 shows the cornea 10 with the ulcerative region 20, and the user interface 132 provides the cursor 132b on the display 132a. According to the alternative approach illustrated in FIG. 5B, the operator can identify a border of the ulcerative region 20 more directly by moving the cursor 132b to produce a tracing 22 (shown as a dashed line) that follows the border. To register the tracing 22 for use by the controller 130, the operator may have to click the button on the computer mouse repeatedly or press the button continuously as the cursor 132b produces the tracing 22. (Alternative user interface tools are also contemplated; for instance, the user interface 132 can provide a touchpad that allows the operator to trace the border, or the user interface 132 can provide a touchscreen that allows the operator to trace the tracing 22 a touchscreen (e.g., with a stylus)).

Once the border of the ulcerative region 20 is identified according to the approach in FIG. 5A or 5B, the controller 130 may employ eye tracking techniques to follow changes in the position of the ulcerative region 20 due to any movement of the cornea 10 relative to the illumination system 120. As such, the illumination system 120 can accurately deliver the illumination 122 with desired parameters to the ulcerative region 20 and other treatment zones around or outside the ulcerative region 20. The eye tracking techniques may employ a series of time-elapsed images captured by the imaging system 134 to determine changes to the position of the ulcerative region 20.

The interaction of a photosensitizing agent, such as riboflavin, and activating illumination, such as UV light, produce at least three factors that produce an antimicrobial effect and that can be used to treat keratitis. In particular, a first antimicrobial factor involves oxygen depletion; a second antimicrobial factor involves generation of singlet oxygen; and a third antimicrobial factor involves generation of hydrogen peroxide. More optimal levels of each factor can be generated, for instance, with different combinations of drug concentration, ambient oxygen level, light source irradiance, and/or treatment time. The levels of each factor in a treatment may depend on a threshold condition for each factor that triggers irreversible apoptosis and cell death. Photosensitizing agents other than riboflavin and light wavelengths other than ultraviolet may be used as well, as long as the light wavelength is chosen to correspond to an efficient optical absorption zone of the photosensitizing agent.

In treatments employing riboflavin as a photosensitizing agent, desired antimicrobial effect may be achieved by employing higher riboflavin concentrations (e.g., 0.5% or 1%) than what are typically used for treating ectatic disorders, such as keratoconus, with corneal cross-linking (e.g., 0.1% to 0.25%). Higher riboflavin concentrations as well as reduced soak times may be achieved, for instance, by using solubility-enhancing excipients, alternate delivery vehicles such as hydrogels, and/or drug-eluting contact lenses. It may also be preferable to avoid the use of ionic surfactants or other irritating additives since the presence of an active infection can significantly increase ocular sensitivity.

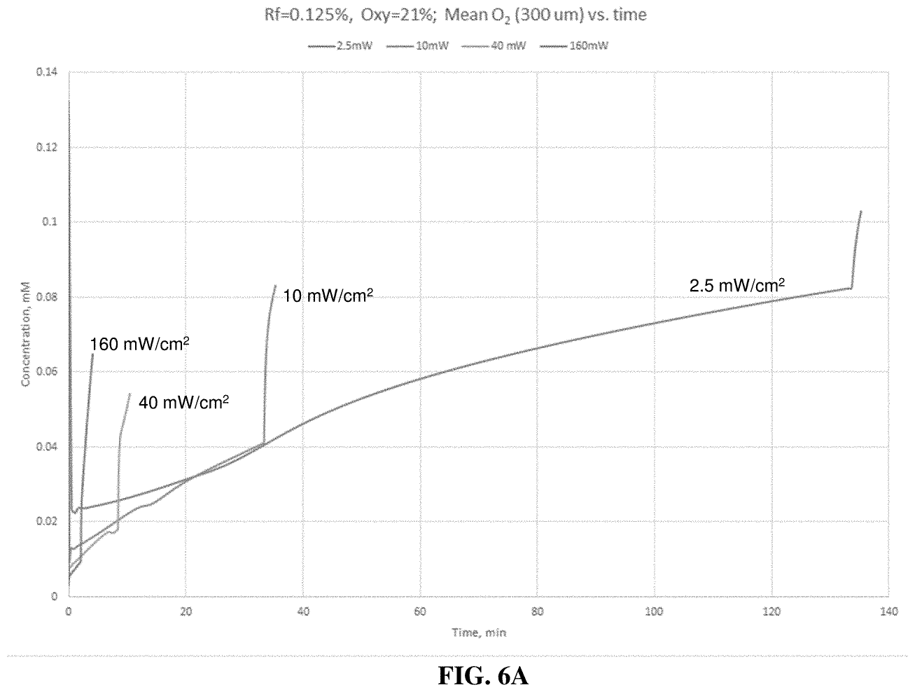

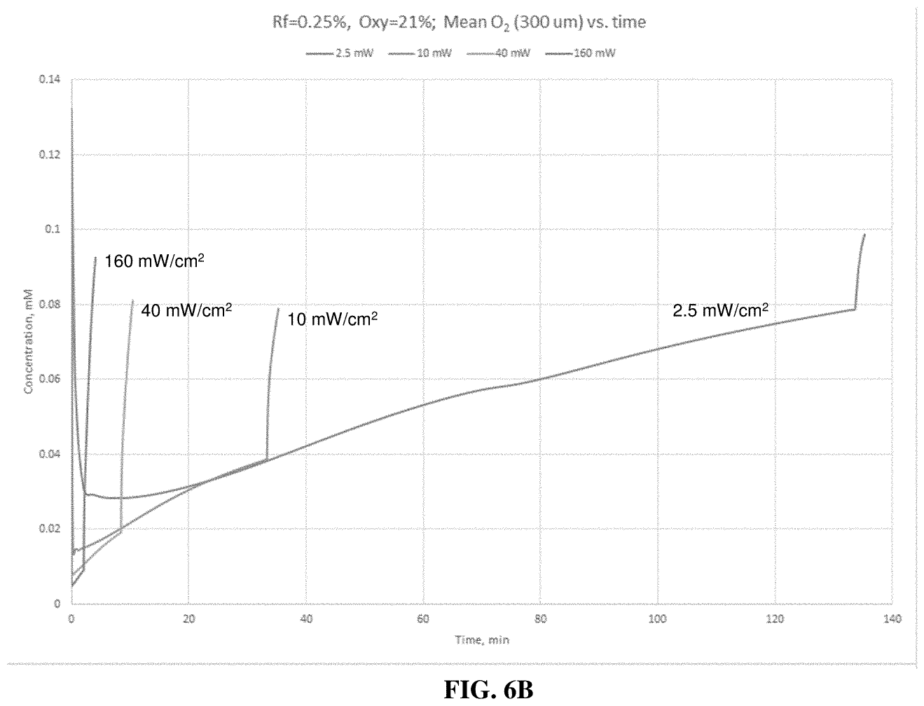

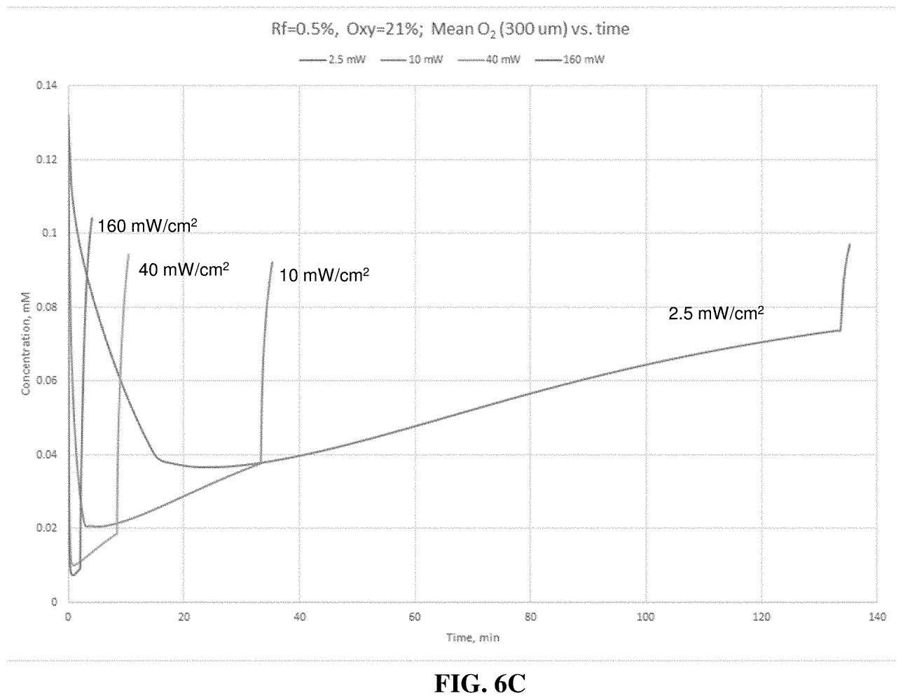

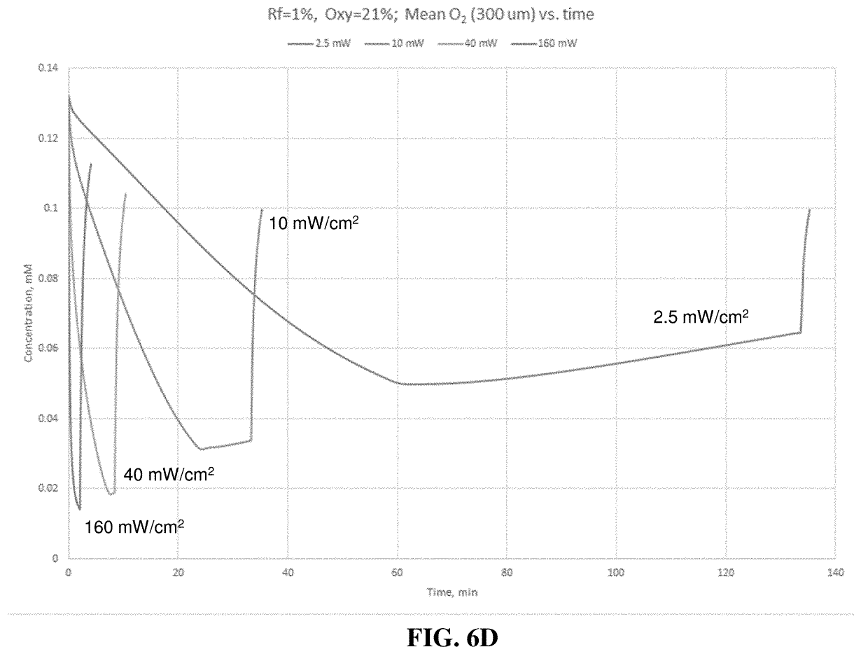

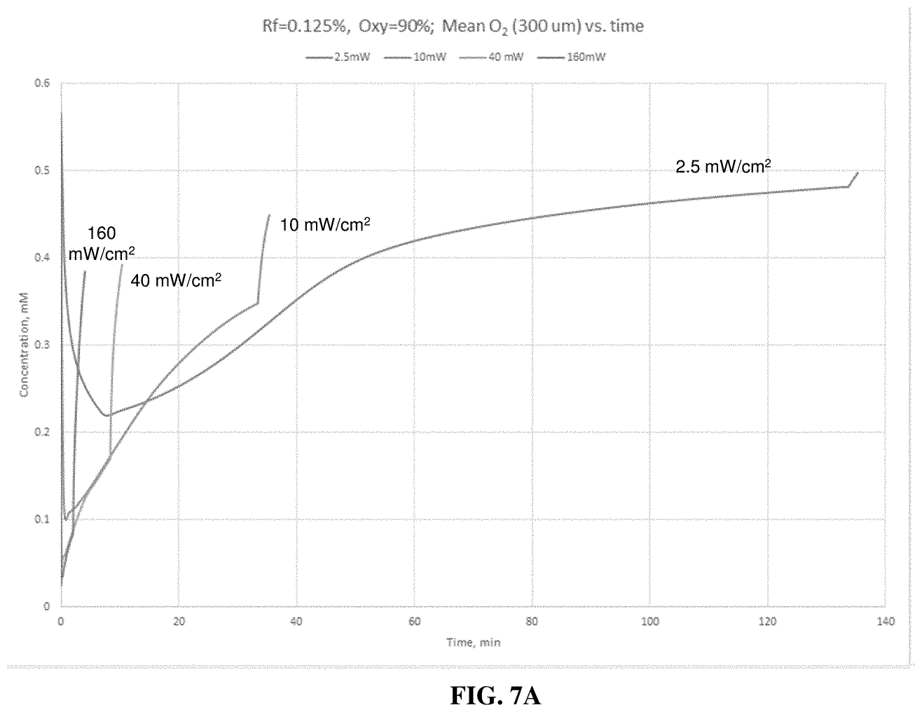

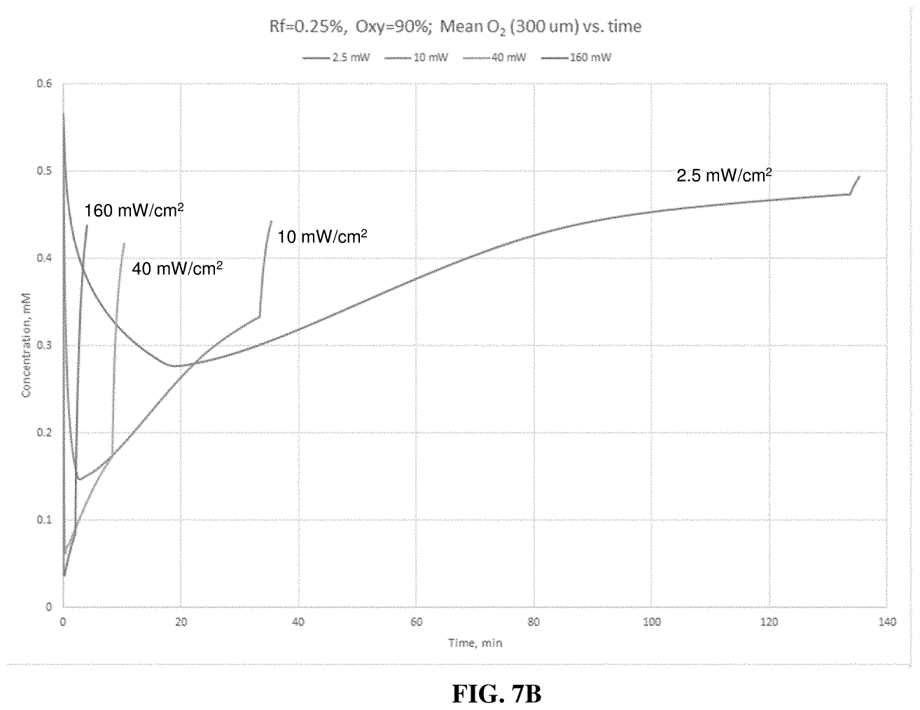

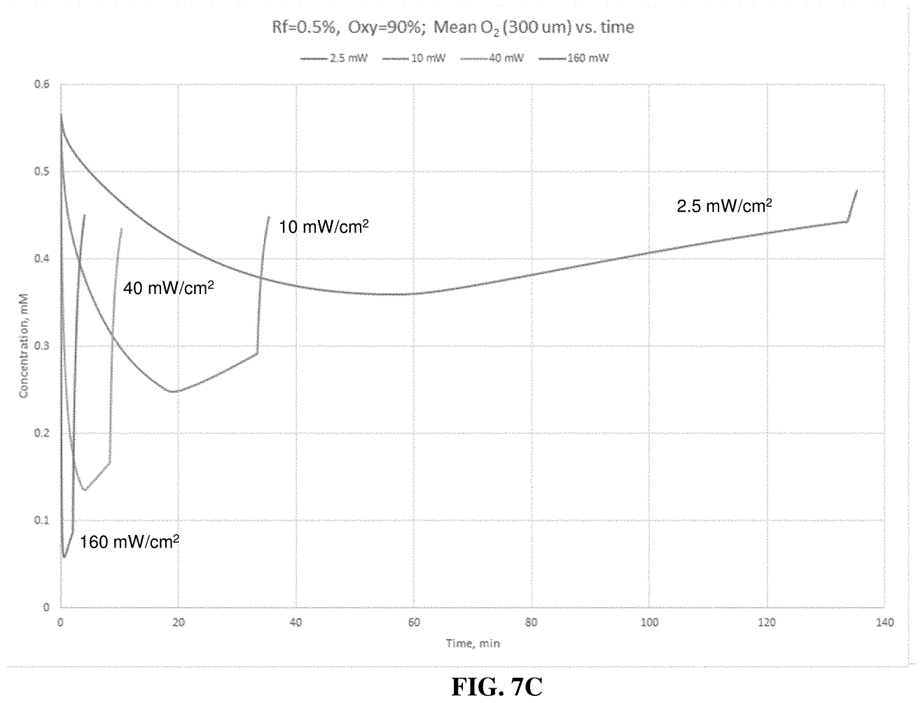

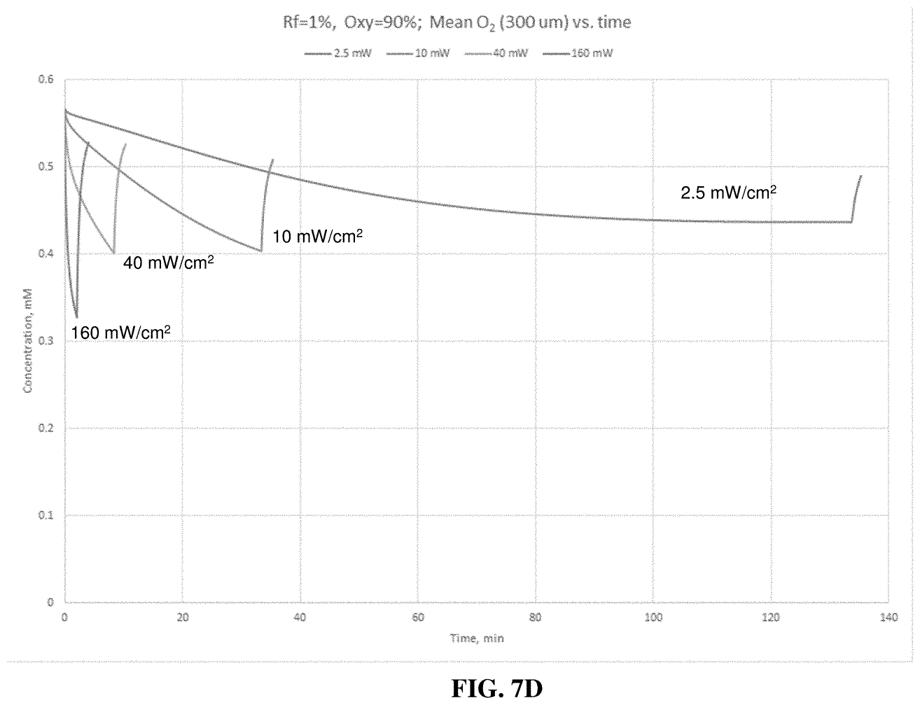

FIGS. 6A-D, 7A-D illustrate the results of studies considering oxygen (O₂) depletion for antimicrobial treatments that deliver varying doses of UV light to activate varying concentrations of riboflavin applied to a cornea under normoxic and hyperoxic conditions. FIGS. 6A-D show mean concentration of O₂ (mM) at a depth of 300 μm in the cornea as a function of time when the cornea is exposed to a concentration of 21% ambient O₂ during treatment (normoxic treatment). Meanwhile, FIGS. 7A-D show mean concentration of O₂ (mM) at a depth of 300 μm in the cornea as a function of time when the cornea is exposed to a concentration of 90% ambient O₂ during treatment (hyperoxic treatment). FIGS. 6A, 7A show results for a riboflavin concentration of 0.125%. FIGS. 6B, 7B show results for a riboflavin concentration of 0.25%. FIGS. 6C, 7C show results for a riboflavin concentration of 0.5%. FIGS. 6D, 7D show results for a riboflavin concentration of 1%. For each riboflavin concentration, the UV light was delivered at each of the irradiances of 2.5 mW/cm², 10 mW/cm², 40 mW/cm², and 160 mW/cm² to activate the riboflavin.

The studies show that the deepest and longest oxygen depletion occurs for normoxic treatments as shown in FIGS. 6A-D. Notably higher O₂ concentrations occur for hyperoxic treatments as shown in FIGS. 7A-D. Among the normoxic treatments, the deepest and longest oxygen depletion occurs at riboflavin concentrations of 0.125% and 0.25% as shown in FIGS. 6A, B, respectively. In general, the studies show that oxygen depletion has a weak dependence on riboflavin concentration. Rapid oxygen replenishment occurs when treatment ends. Larger irradiances result in deeper oxygen depletion and faster oxygen replenishment, though the effect is smaller for normoxic treatment. Increasing irradiance beyond 40 mW/cm² does not make oxygen depletion deeper for normoxic treatment.

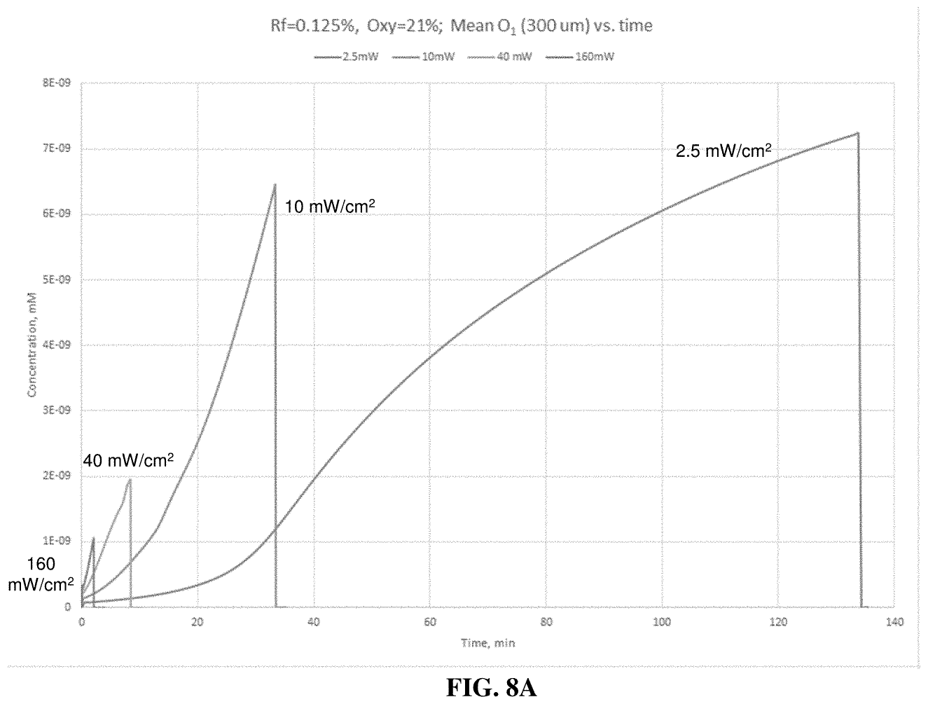

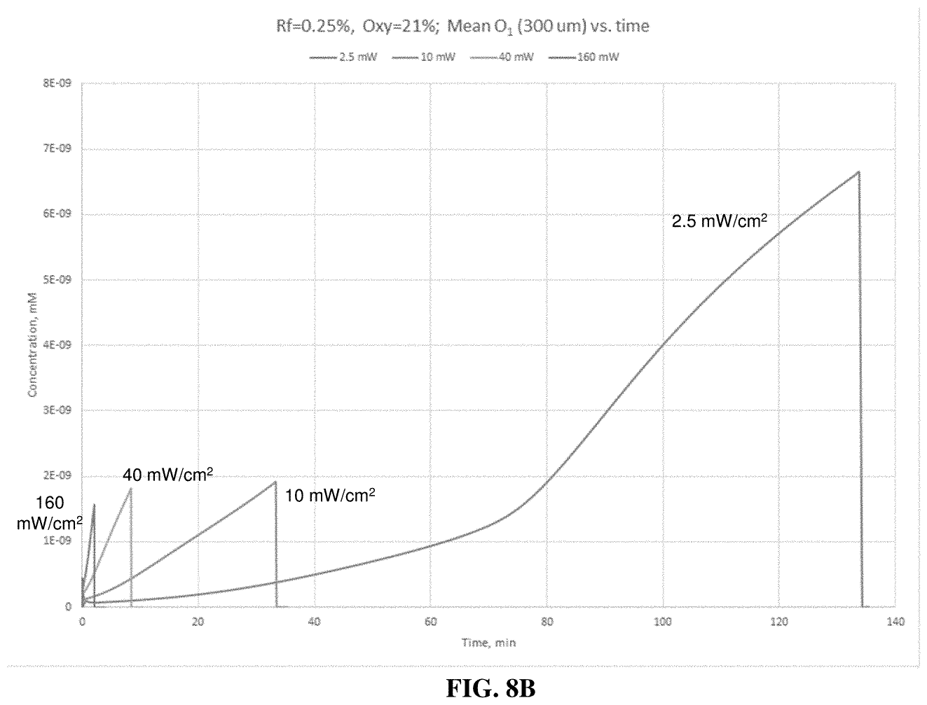

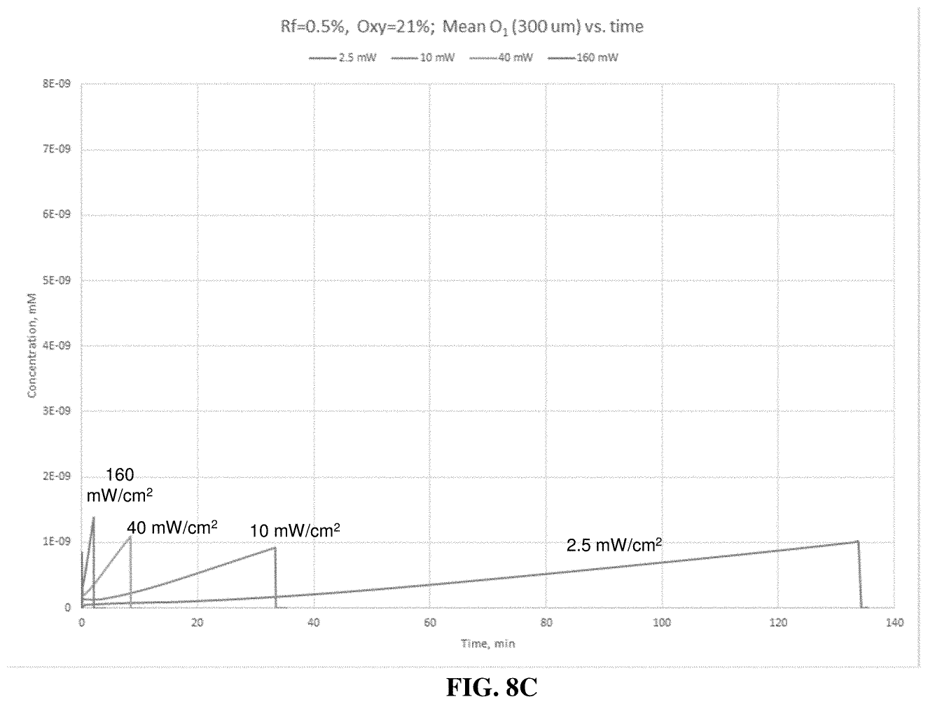

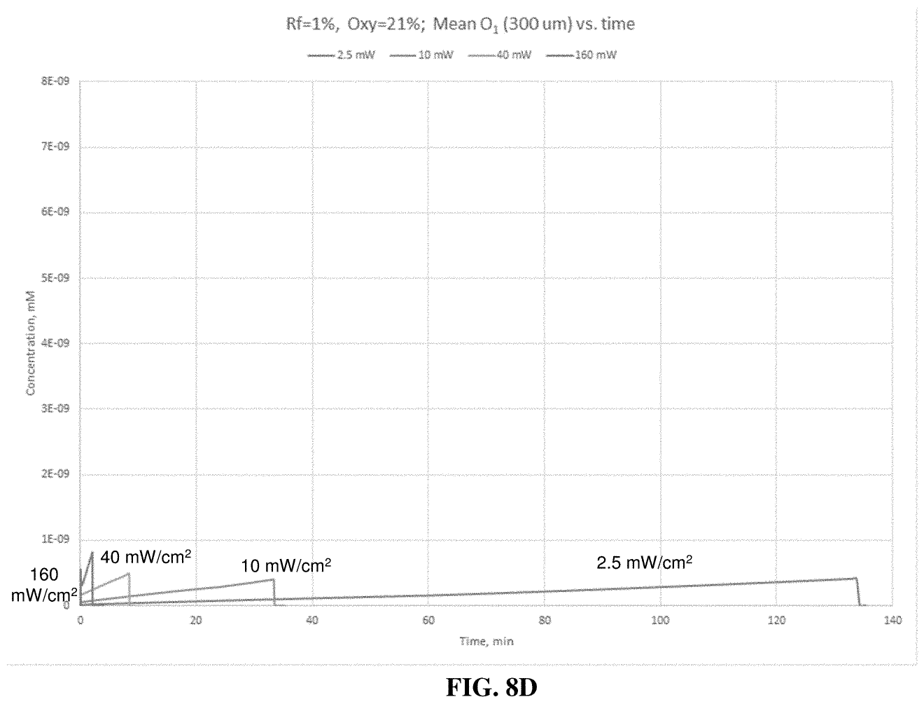

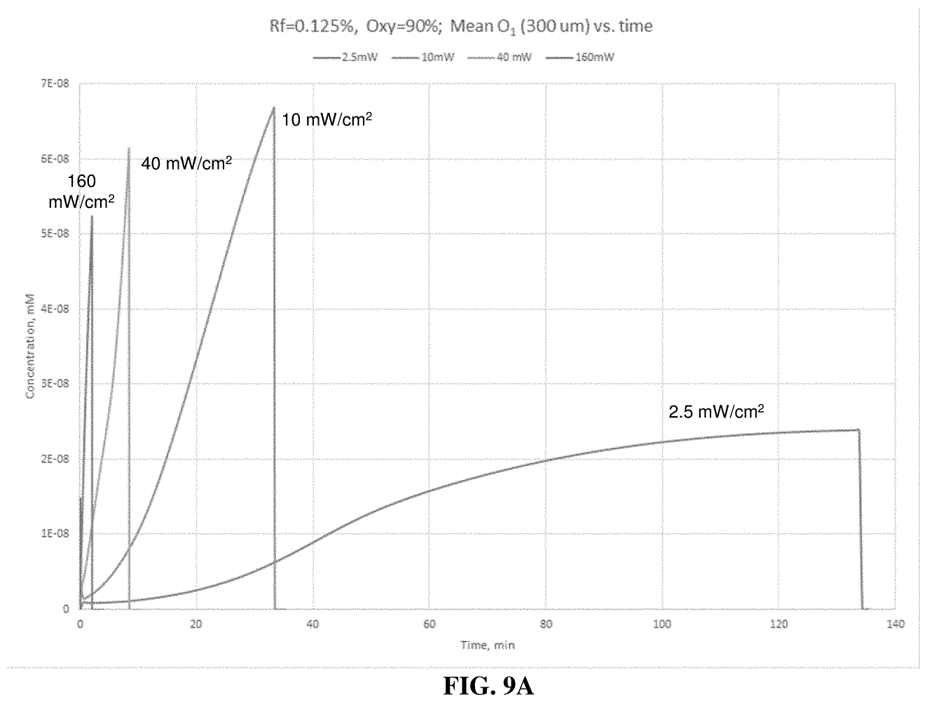

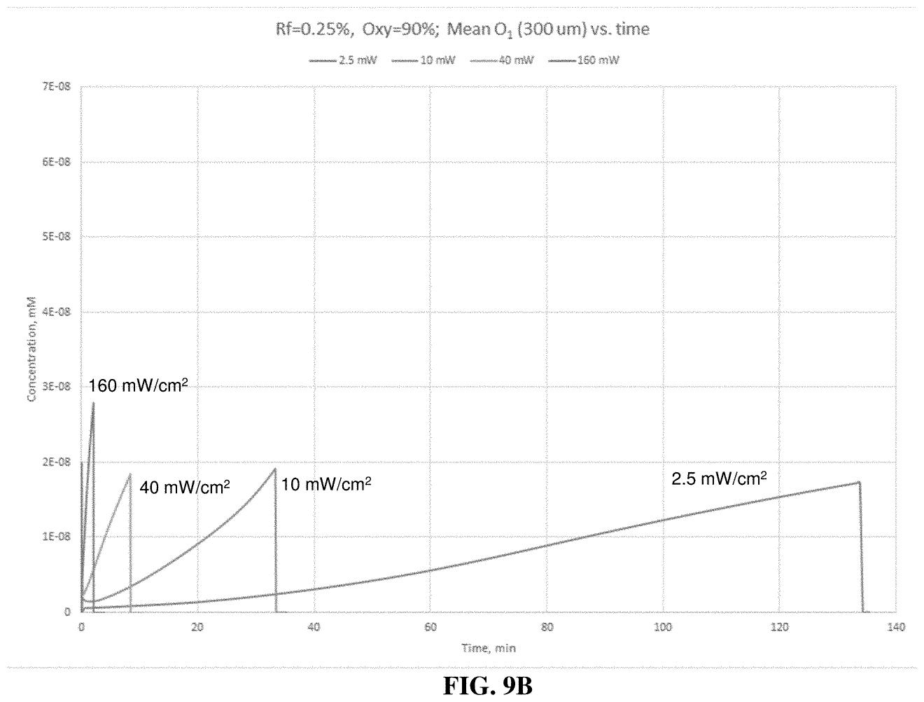

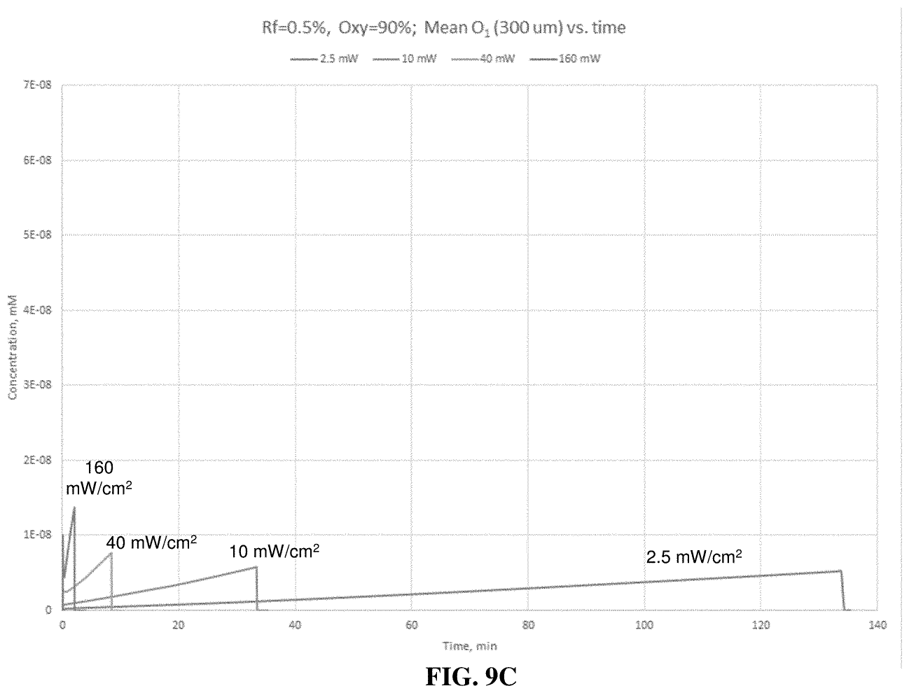

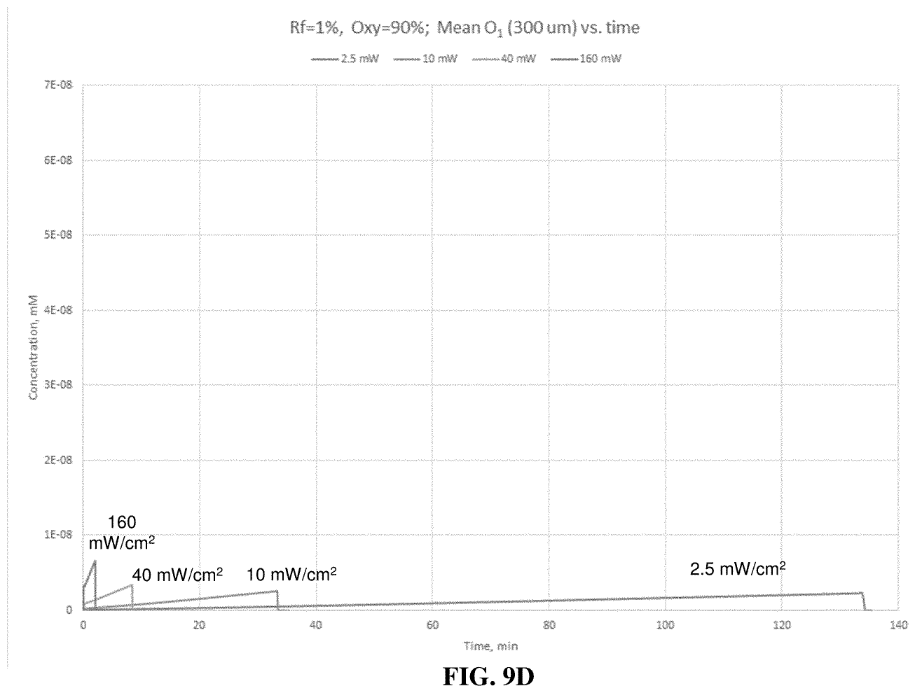

FIGS. 8A-D, 9A-D illustrate the results of studies considering generation of singlet oxygen (O₁) for antimicrobial treatments that deliver varying doses of UV light to activate varying concentrations of riboflavin applied to a cornea under normoxic and hyperoxic conditions. FIGS. 8A-D show mean concentration of O₁ (mM) at a depth of 300 μm in the cornea as a function of time when the cornea is exposed to a concentration of 21% ambient O₂ during treatment (normoxic treatment). Meanwhile, FIGS. 9A-D show mean concentration of O₁ (mM) at a depth of 300 μm in the cornea as a function of time when the cornea is exposed to a concentration of 90% ambient O₂ during treatment (hyperoxic treatment). FIGS. 8A, 9A show results for a riboflavin concentration of 0.125%. FIGS. 8B, 9B show results for a riboflavin concentration of 0.25%. FIGS. 8C, 9C show results for a riboflavin concentration of 0.5%. FIGS. 8D, 9D show results for a riboflavin concentration of 1%. For each riboflavin concentration, the UV light was delivered at each of the irradiances of 2.5 mW/cm², 10 mW/cm², 40 mW/cm², and 160 mW/cm² to activate the riboflavin.

The studies show that the generation of singlet oxygen is significantly higher in the hyperoxic treatments as shown in FIGS. 9A-D. Among the hyperoxic treatments, generation of singlet oxygen is most efficient at a riboflavin concentration of 0.125% as shown in FIG. 9A. The studies also show that the generation of singlet oxygen has a strong inverse dependence on the concentration of riboflavin. Additionally, the studies show that the rate of generation of singlet oxygen depends strongly on irradiance. Increasing irradiance from 2.5 mW/cm² to 160 mW/cm² results in a sharp rise in the generation of singlet oxygen. When illumination is applied, the concentration of singlet oxygen increases linearly as a function of time for higher irradiances of 10 mW/cm² and more. For lower irradiance of 2.5 mW/cm², the concentration of singlet oxygen stabilizes after about 100 min. Because singlet oxygen is an unstable component, the concentration of singlet oxygen drops rapidly when the illumination is ended. Treatments based on the generation of singlet oxygen may consider further reactions with the singlet oxygen, such as destruction of riboflavin by singlet oxygen.

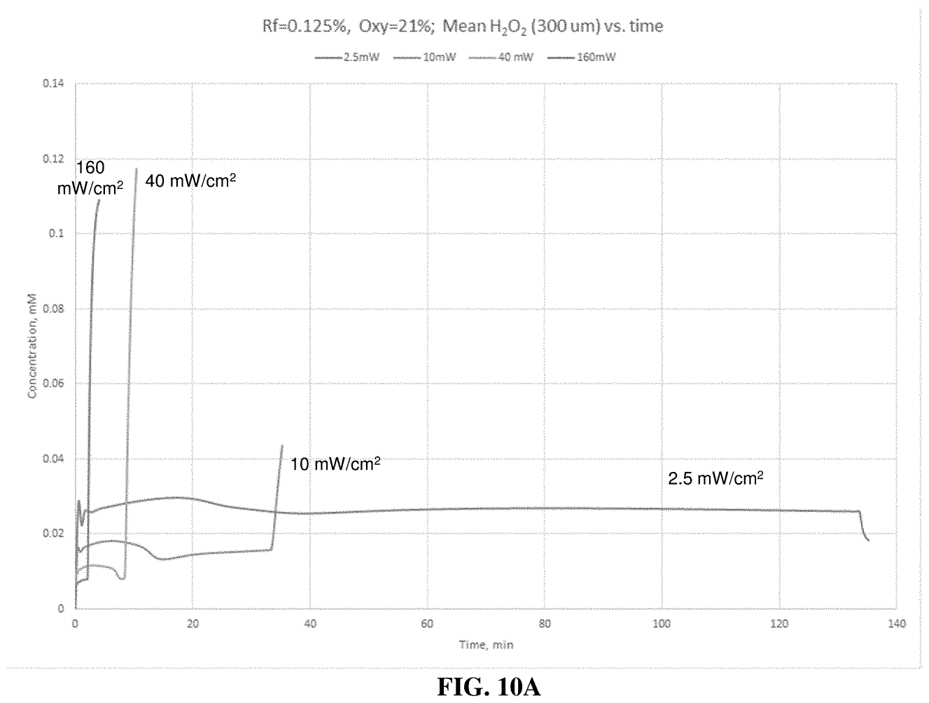

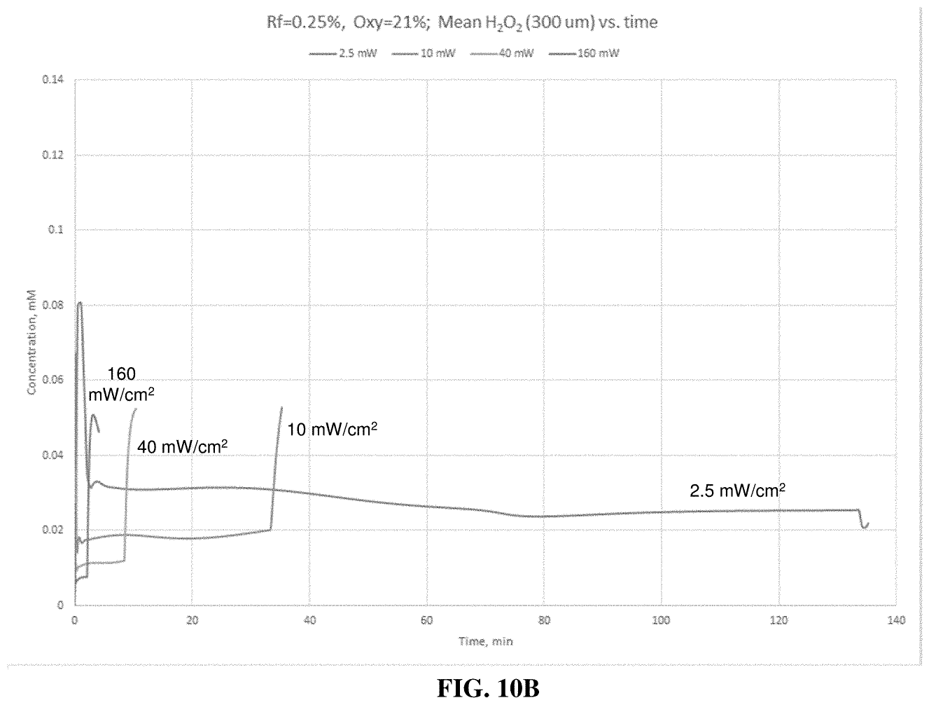

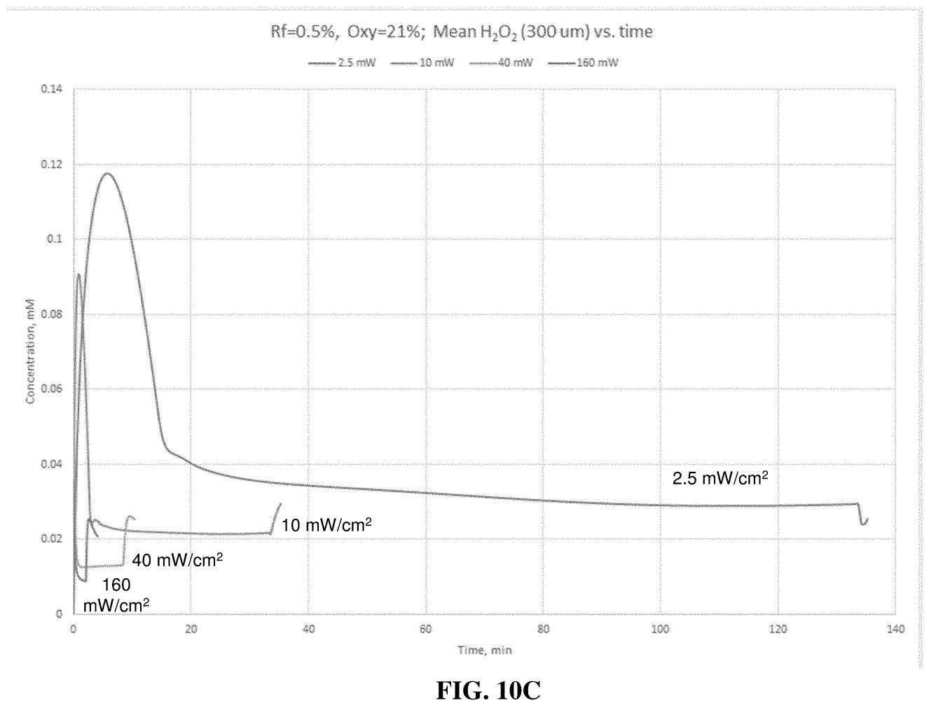

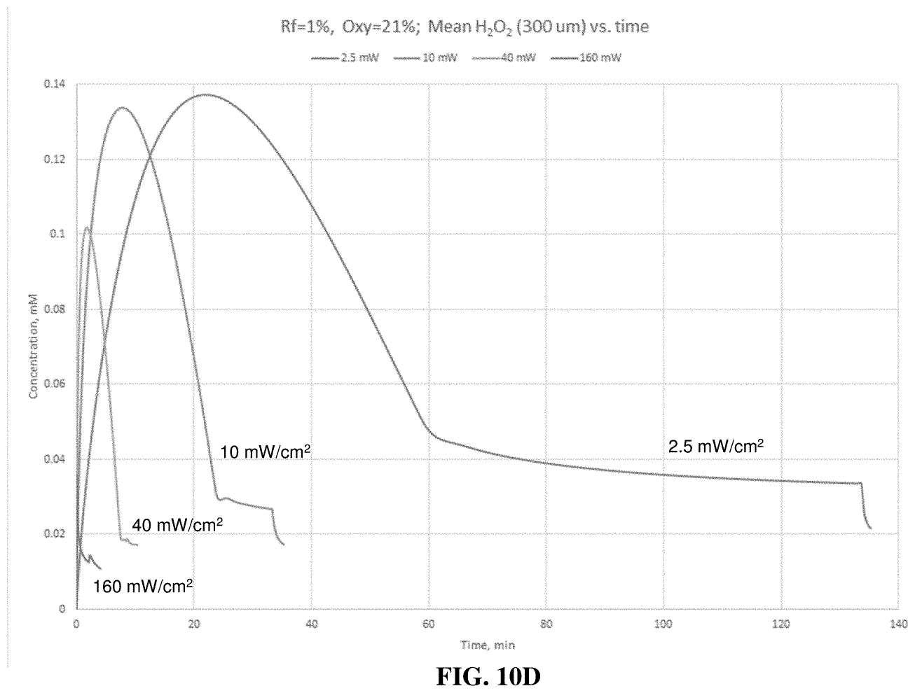

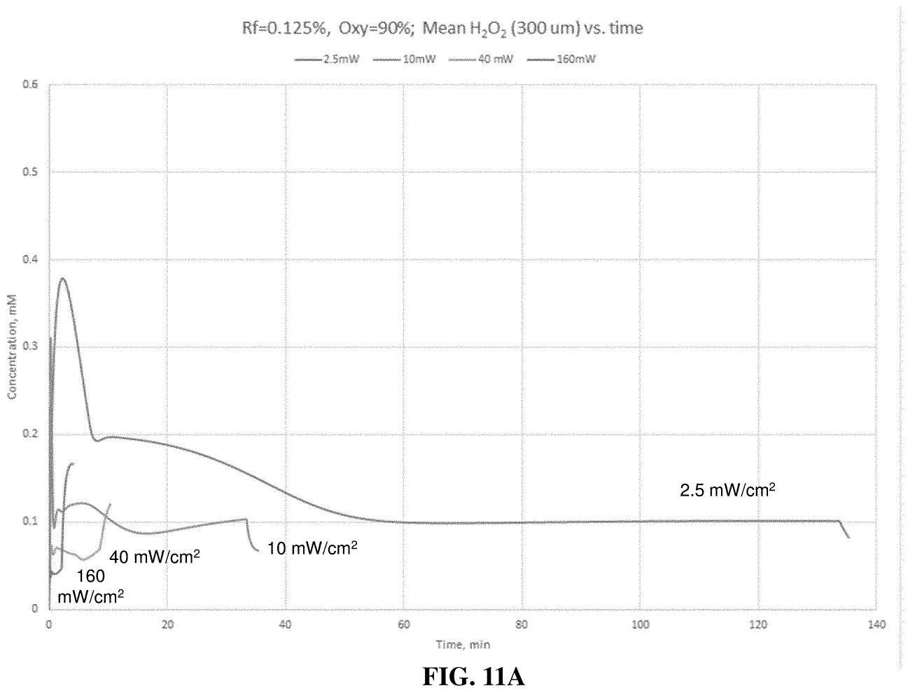

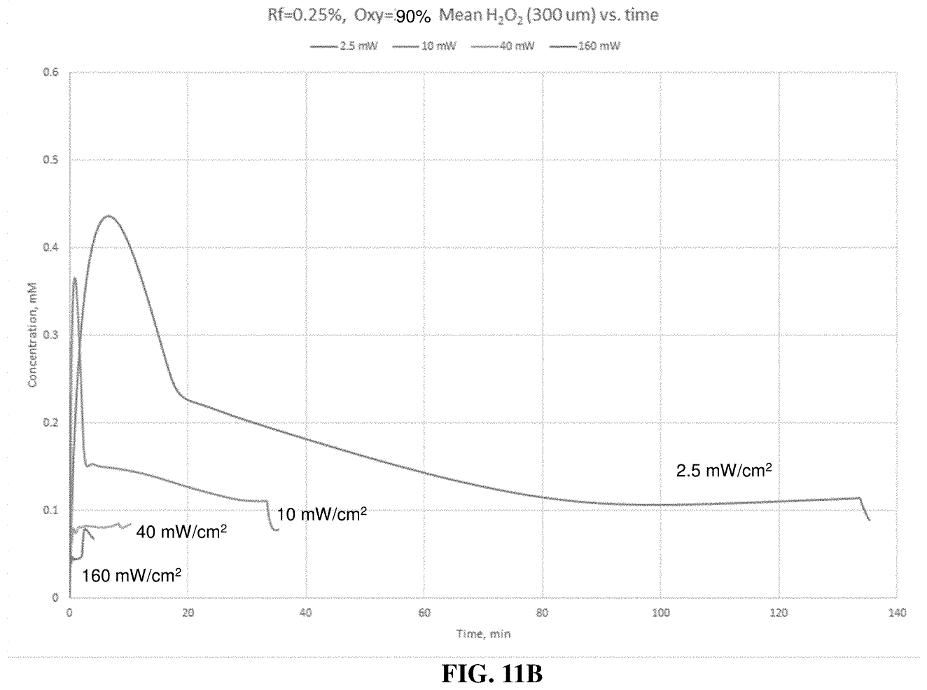

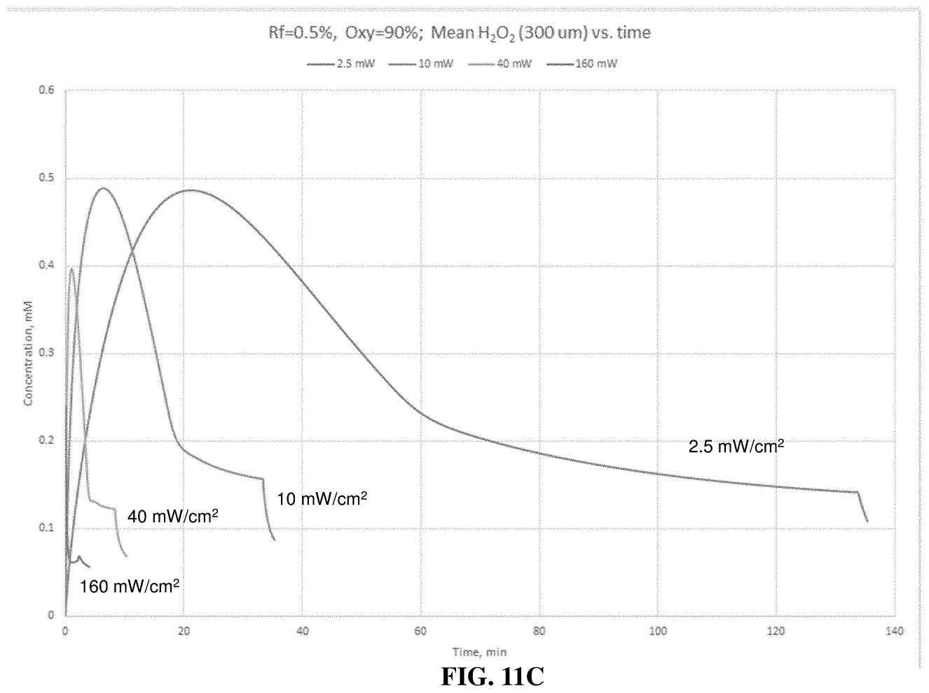

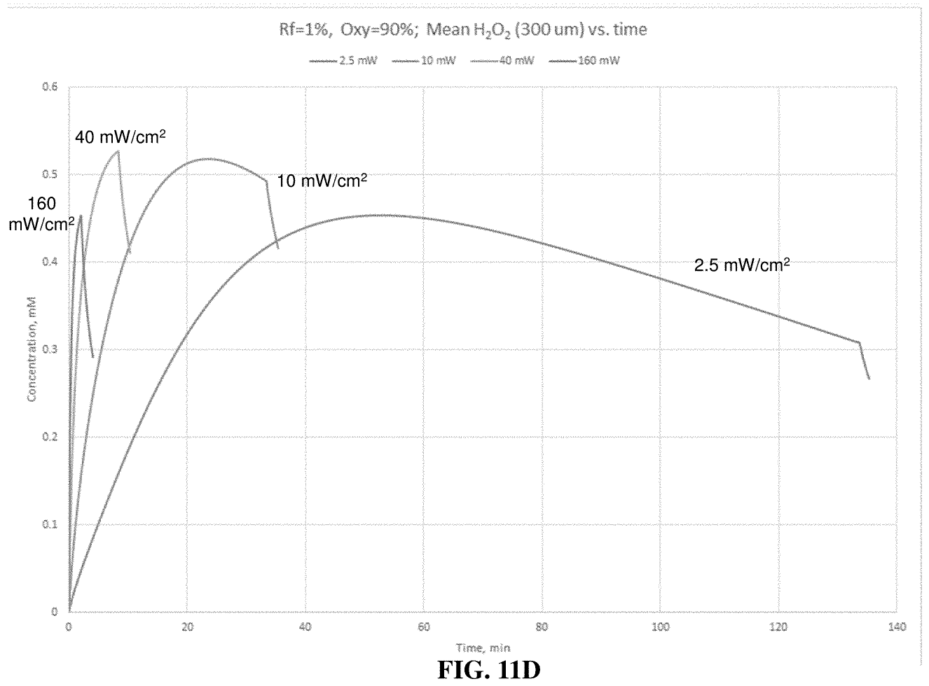

FIGS. 10A-D, 11A-D illustrate the results of studies considering generation of hydrogen peroxide (H₂O₂) for antimicrobial treatments that deliver varying doses of UV light to activate varying concentrations of riboflavin applied to a cornea under normoxic and hyperoxic conditions. FIGS. 10A-D show mean concentration of H₂O₂ (mM) at a depth of 300 μm in the cornea as a function of time when the cornea is exposed to a concentration of 21% ambient O₂ during treatment (normoxic treatment). Meanwhile, FIGS. 11A-D show mean concentration of H₂O₂ (mM) at a depth of 300 μm in the cornea as a function of time when the cornea is exposed to a concentration of 90% ambient O₂ during treatment (hyperoxic treatment). FIGS. 10A, 11A show results for a riboflavin concentration of 0.125%. FIGS. 10B, 11B show results for a riboflavin concentration of 0.25%. FIGS. 10C, 11C show results for a riboflavin concentration of 0.5%. FIGS. 10D, 11D show results for a riboflavin concentration of 1%. For each riboflavin concentration, the UV light was delivered at each of the irradiances of 2.5 mW/cm², 10 mW/cm², 40 mW/cm², and 160 mW/cm² to activate the riboflavin.

The studies show that the generation of hydrogen peroxide is significantly higher in hyperoxic treatments as shown in FIGS. 11A-D. Among the hyperoxic treatments, generation of hydrogen peroxide is most efficient at a riboflavin concentration of 1% as shown in FIG. 11D. The studies also show that upon the delivery of the UV light, the concentration of hydrogen peroxide increases to a peak value and the decreases. The highest peak concentration of hydrogen peroxide (within the design of experiment (DOE) range) occurs at a riboflavin concentration of 1% under irradiances of 10 mW/cm² to 40 mW/cm² as shown in FIGS. 10D, 11D. Increasing irradiance beyond 40 mW/cm² reduces the peak concentration of hydrogen peroxide. The concentration of hydrogen peroxide drops rapidly after treatment is completed. Treatments based on the generation of hydrogen peroxide may consider the lifetime of hydrogen peroxide after illumination is ended.

Oxygen depletion, generation of singlet oxygen, and generation of hydrogen peroxide are antimicrobial factors that can be optimized based on various treatment parameters, such as drug concentration, ambient oxygen level, irradiance of activating illumination, and/or treatment time. In view of the results of the studies above, greater antibacterial effect based on oxygen depletion can be achieved with a normoxic treatment (21% oxygen concentration) with a lower riboflavin concentration of 0.125% under moderate irradiance of 40 mW/cm². Greater antibacterial effect based on generation of singlet oxygen can be achieved with a hyperoxic treatment (90% oxygen concentration) with a lower riboflavin concentration of 0.125% under higher irradiance of 160 mW/cm². Greater antibacterial effect based on generation of hydrogen peroxide can be achieved with a hyperoxic treatment (90% oxygen concentration) with a higher riboflavin concentration of 1% under moderate irradiances of 10 mW/cm² to 40 mW/cm².

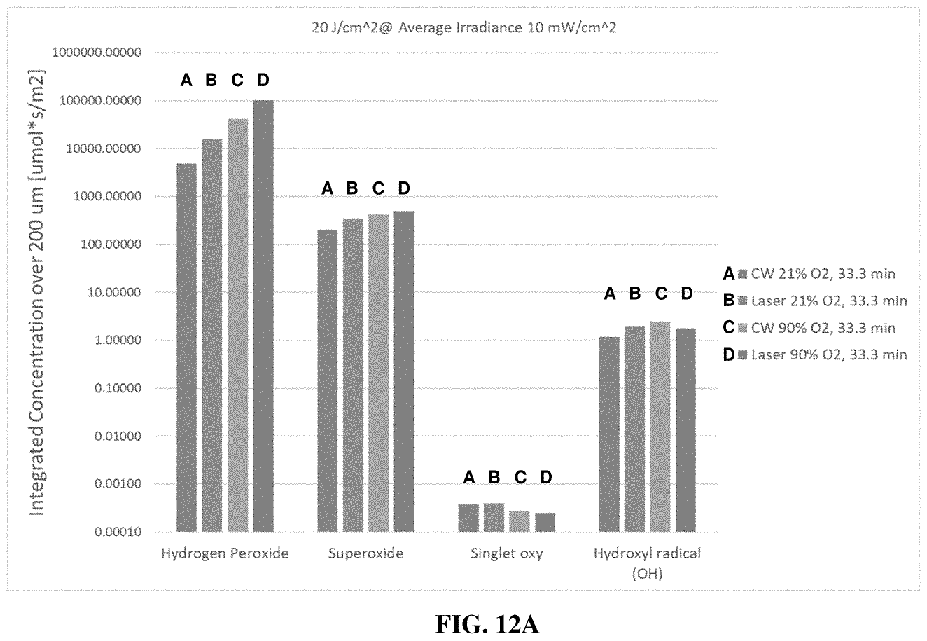

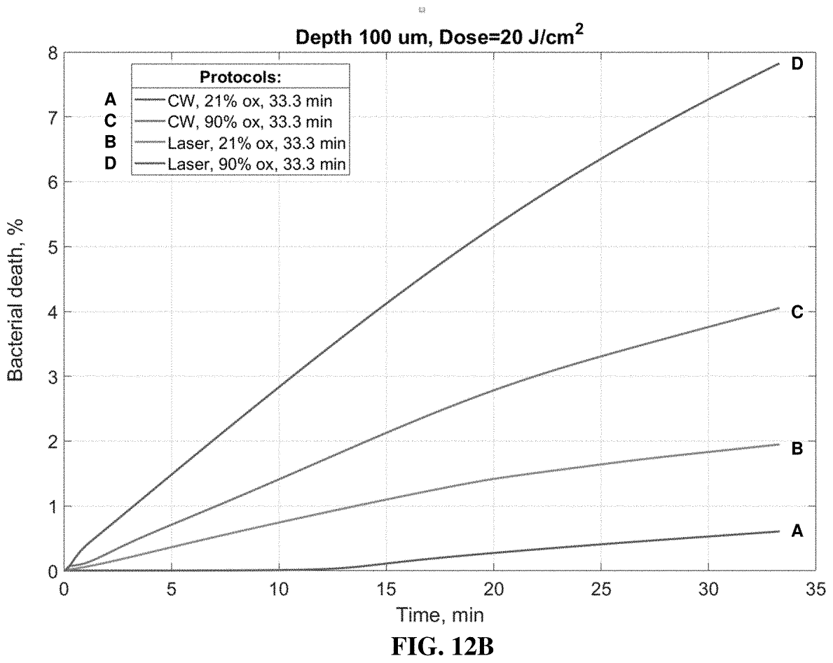

FIGS. 12A, B illustrate modelling of bacterial death for treatment protocols A-D employing riboflavin as a photosensitizing agent. Protocol A applies continuous wave (CW) UV light from an LED for 33.3 minutes while the eye is exposed to an oxygen (O₂) concentration of 21%. Protocol B applies a laser beam of UV light for 33.3 minutes while the eye is exposed to an O₂ concentration of 21%. Protocol C applies CW UV light from an LED for 33.3 minutes while the eye is exposed to an O₂ concentration of 90%. Protocol D applies a laser beam of UV light for 33.3 minutes while the eye is exposed to an O₂ concentration of 90%. The protocols A-D deliver a dose of approximately 20 J/cm² at an average irradiance of approximately 10 mW/cm². The graph of FIG. 12A shows predicted production of H₂O₂ and reactive oxygen species (ROS) (i.e., superoxide, singlet oxygen, and hydroxyl radical (OH)) for each protocol. The predicted production is expressed as an integrated concentration over approximately 200 μm into the cornea. Meanwhile, the graph of FIG. 12B shows bacterial death rates for each protocol at a depth of approximately 100 μm in the cornea.

FIGS. 12A, B show, that despite lower toxicity than ROS, H₂O₂ plays a significant role in bacterial death due to higher predicted concentrations. Additionally, FIG. 12B shows that the laser beam of UV light (protocols B and D) produces greater bacterial death than CW UV light from an LED (protocols A and C) by a factor of approximately two to three, and that the O₂ concentration of 90% (protocols C and D) produces greater bacterial death than normoxic conditions (protocols A and B) by a factor of approximately four to eight.

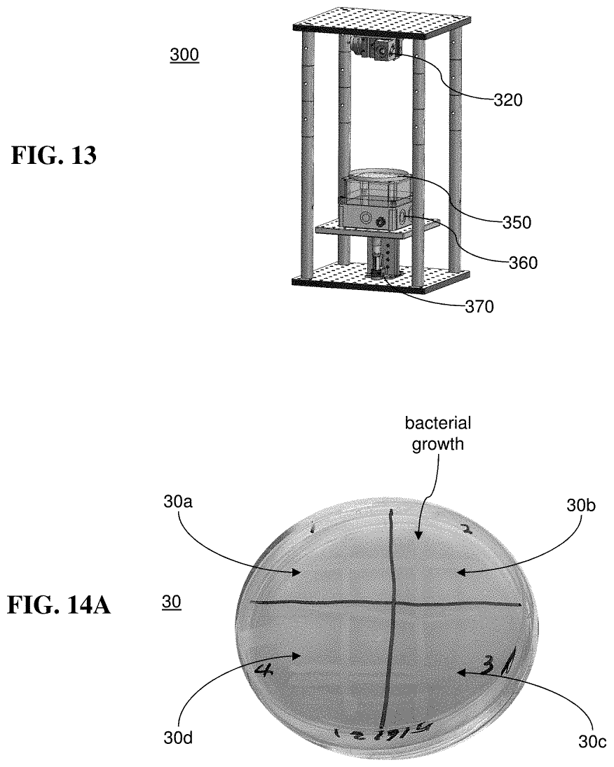

FIG. 13 illustrates an example system 300 for an in vitro study of keratitis treatments. The system 300 includes an illumination system 320 that emits a laser beam that can be scanned across a petri dish containing a sample. The system 300 also includes a UV window 350 that can be selectively employed to transmit UV light in the study. Additionally, the system 300 includes a holder 360 that holds the petri dish. The holder 360 includes tubing to receive and deliver a concentration of O₂ to some samples in the study. Furthermore, the system 300 includes a micrometer 370 to determine the position of the holder 360 and thus the petri dish along a vertical axis (z-axis) relative to the illumination system 320.

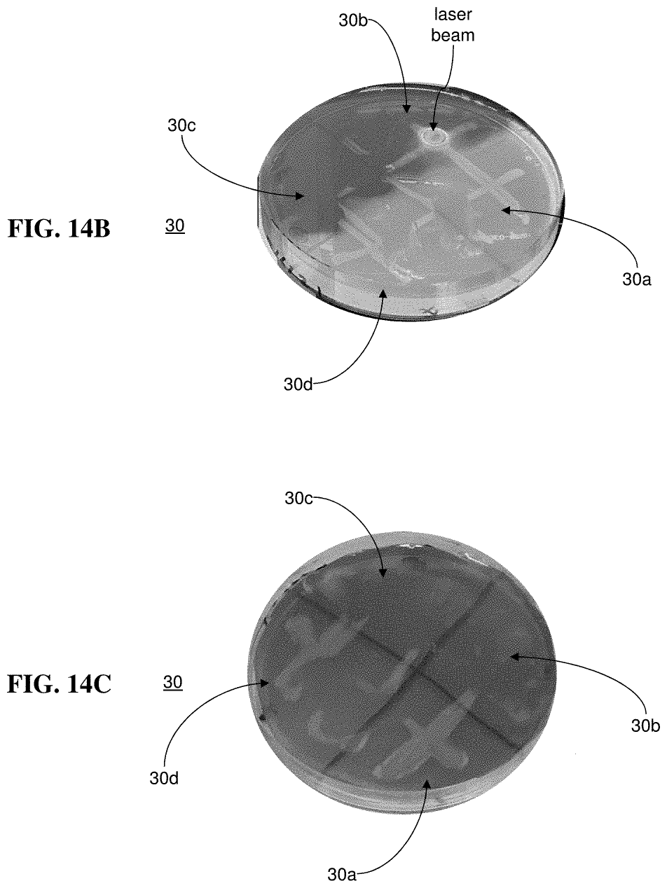

FIG. 14A illustrates a petri dish with an example sample 30 for the in vitro study of keratitis treatments employing the system 300. The preparation of the sample 30 involves inoculation with Pseudomonas aeruginosa bacteria (which causes keratitis) and incubation for approximately 48 hours. The resulting bacterial growth in the sample 30 is visible in FIG. 14A. The sample 30 is divided into four quadrants 30a-d to mark areas of the bacterial growth that are exposed to different conditions. In particular, the first quadrant 30a receives UV light only; the second quadrant 30b and the third quadrant 30c both receive a riboflavin composition and photoactivating UV light at a dose of approximately 4.5 J/cm² and an irradiance of approximately 9 mW/cm²; and the fourth quadrant 30d receives neither riboflavin nor UV light.

FIG. 14B illustrates the delivery of the laser beam from the illumination system 320 to selected quadrants of the sample 30. As shown, the laser beam is scanning the second quadrant 30b. FIG. 14C illustrates results approximately 70 hours after the quadrants 30a-d of the sample 30 have been exposed to different conditions. The bacteria has been effectively eliminated in the second quadrant 30b and the third quadrant 30c, both of which received riboflavin and photoactivating UV light. Meanwhile, the bacteria generally remains in the first quadrant 30a which only receives UV light and the fourth quadrant 30d which receives neither riboflavin nor UV light.

The sample 30 in FIG. 14B is treated in ambient air (i.e., no concentration of O₂). Further aspects of the study, however, may involve considerations of different concentrations of O₂ as well as different illumination systems (e.g., LED), different illumination parameters, different photosensitizing agents/drugs; and/or different doses of photosensitizing agents/drugs.

Studies by the present inventors indicate that the antimicrobial effect depends on UV dose and power. Such studies also indicate that the riboflavin solution on its own (without photoactivation) does not directly inhibit bacterial growth.

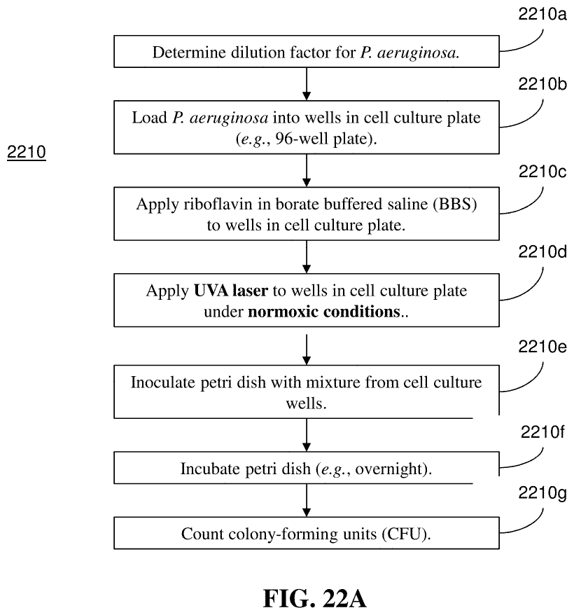

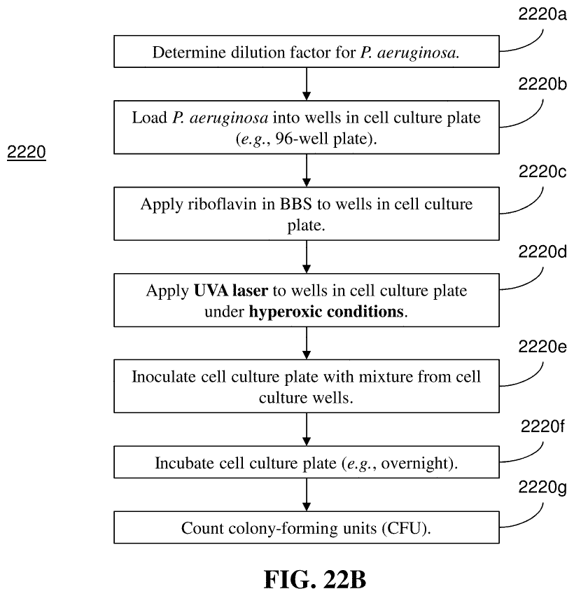

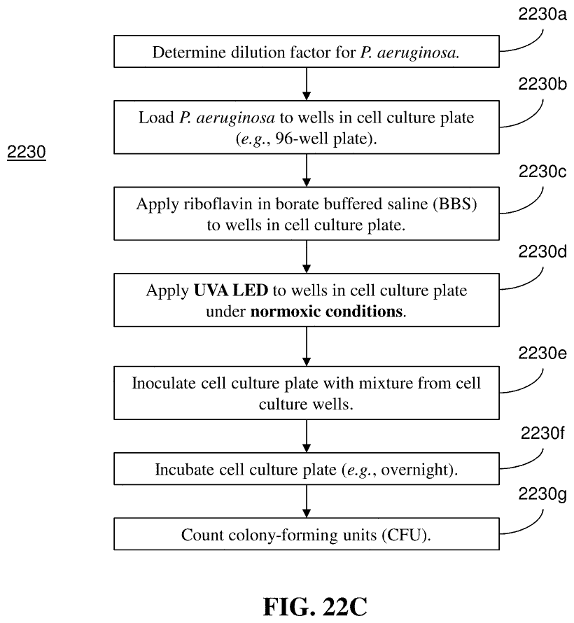

FIGS. 22A-C illustrate example procedures for studying the effects of riboflavin/UVA treatments on Pseudomonas aeruginosa bacteria. According to an example procedure 2210 shown in FIG. 22A, a dilution factor is determined for Pseudomonas aeruginosa bacteria in step 2210a, and the Pseudomonas aeruginosa is loaded into wells in cell culture plate (e.g., a 96-well plate) in step 2210b. According to particular treatment parameters, a concentration of riboflavin in borate buffered saline (BBS) is applied to the wells in step 2210c, and ultraviolet light is applied to the wells with a laser beam at a specified irradiance and dose under normoxic conditions (a concentration of 21% ambient O₂) in step 2210d. In particular, a laser beam of ultraviolet A (UVA) light can be applied with a size of approximately 6.5 mm at 8 Hz. A petri dish is inoculated with the mixture from the wells in step 2210e and incubated (e.g., overnight) in step 2210f. After incubation, the effect of the particular treatment parameters is evaluated by counting colony forming units (CFU) in step 2210g.

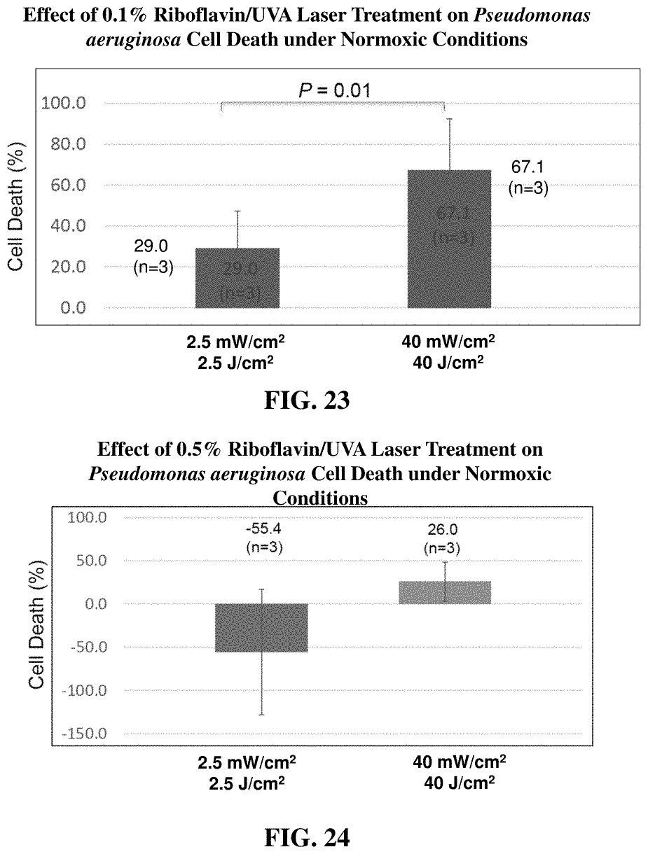

In a study employing the example procedure 2210, a riboflavin concentration of 0.1% was applied and photoactivated with a UVA laser at an irradiance of 2.5 mW/cm² and a dose of 2.5 J/cm² (applied for 16 minutes, 40 seconds) under normoxic conditions. Additionally, a riboflavin concentration of 0.1% was applied and alternatively photoactivated with a UVA laser light at an irradiance of 40 mW/cm² and a dose of 40 J/cm² (applied for 16 minutes, 40 seconds) under normoxic conditions. TABLE 1 shows the results of this study against a control (growth of Pseudomonas aeruginosa without application of riboflavin and UVA light) for three replicates (n=3). FIG. 23 illustrates a graph indicating an average cell death percentage of 29.0% when the irradiance of 2.5 mW/cm² and the dose of 2.5 J/cm² are applied, and an average cell death percentage of 67.1% when the irradiance of 40 mW/cm² and the dose of 40 J/cm² are applied.

TABLE 1
0.1% Riboflavin Concentration, UVA
Laser under Normoxic Conditions

| | Control | A | B |
|---|---|---|---|
| Irradiance (mW/cm²) | — | 2.5 | 40 |
| Dose (J/cm²) | — | 2.5 | 40 |
| CFU - Replicate 1 | 144 | 91 | 32 |
| CFU - Replicate 2 | 99 | 91 | 61 |
| CFU - Replicate 3 | 142 | 82 | 21 |

In a further study employing the example procedure 2210, a riboflavin concentration of 0.5% was applied and photoactivated with a UVA laser at an irradiance of 2.5 mW/cm² and and a dose of 2.5 J/cm² (applied for 16 minutes, 40 seconds) under normoxic conditions. Additionally, a riboflavin concentration of 0.5% was applied and alternatively photoactivated at an irradiance of 40 mW/cm² and and a dose of 40 J/cm² (applied for for 16 minutes, 40 seconds) under normoxic conditions. TABLE 2 shows the results of this study against a control (growth of Pseudomonas aeruginosa without application of riboflavin and UVA light) for three replicates (n=3). FIG. 24 illustrates a graph indicating an average cell death percentage of −55.4% when the irradiance of 2.5 mW/cm² and the dose of 2.5 J/cm² are applied, and an average cell death percentage of 26.0% when the irradiance of 40 mW/cm² and the dose of 40 J/cm² are applied.

TABLE 2
0.5% Riboflavin Concentration, UVA
Laser under Normoxic Conditions

| | Control | A | B |
|---|---|---|---|
| Irradiance (mW/cm²) | — | 2.5 | 40 |
| Dose (J/cm²) | — | 2.5 | 40 |
| CFU - Replicate 1 | 58 | 51 | 35 |
| CFU - Replicate 2 | 59 | 86 | 37 |
| CFU - Replicate 3 | 37 | 86 | 37 |

According to an example procedure 2220 shown in FIG. 22B, a dilution factor is determined for Pseudomonas aeruginosa bacteria in step 2220a, and the Pseudomonas aeruginosa is loaded into wells in cell culture plate (e.g., a 96-well plate) in step 2220b. According to particular treatment parameters, a concentration of riboflavin in BBS is applied to the wells in step 2220c, and ultraviolet light is applied to the wells with a laser beam at a specified irradiance and dose under hyperoxic conditions (greater than or equal to a concentration of 90% ambient O₂) in step 2220d. In particular, a laser beam of UVA light can be applied with a size of approximately 6.5 mm at 8 Hz. A petri dish is inoculated with the mixture from the wells in step 2220e, and the petri dish is incubated (e.g., overnight) in step 2220f. After incubation, the effect of the treatment parameters is evaluated by counting CFU in step 2220g.

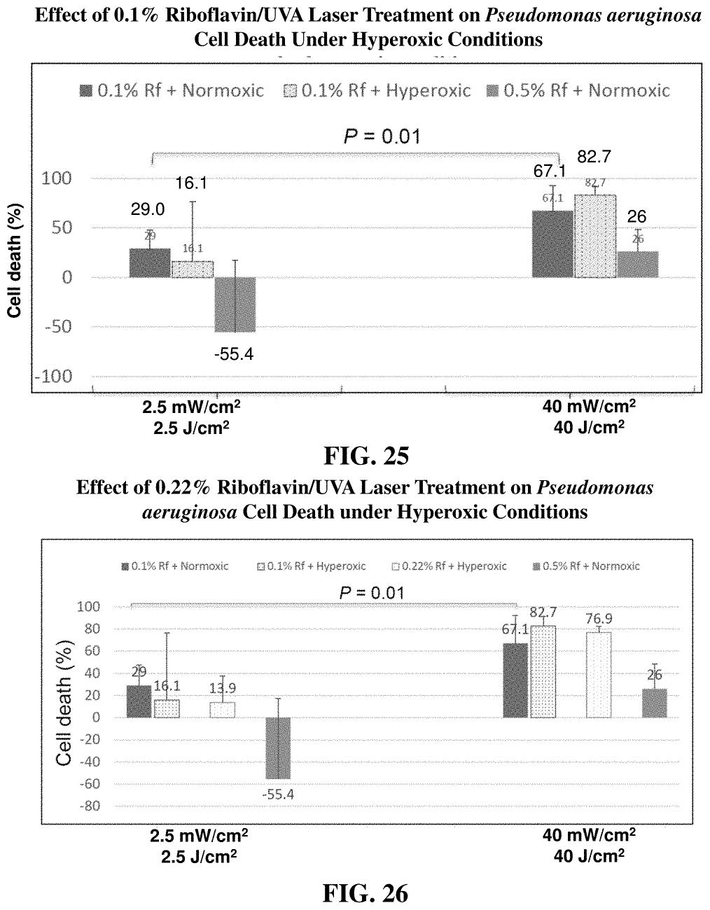

In a study employing the example procedure 2220, a riboflavin concentration of 0.1% was applied and photoactivated with a UVA laser at an irradiance of 2.5 mW/cm² and and a dose of 2.5 J/cm² (applied for 16 minutes, 40 seconds) under hyperoxic conditions. Additionally, a riboflavin concentration of 0.1% was applied and alternatively photoactivated with a UVA laser at an irradiance of 40 mW/cm² and a dose of 40 J/cm² (applied for 16 minutes, 40 seconds) under hyperoxic conditions. TABLE 3 shows the results of this study against controls (growth of Pseudomonas aeruginosa under normoxic conditions and hyperoxic conditions without application of riboflavin and UVA light) for three replicates (n=3). FIG. 25 illustrates a graph indicating an average cell death percentage of 16.1% when the irradiance of 2.5 mW/cm² and the dose of 2.5 J/cm² are applied under hyperoxic conditions, and an average cell death percentage of 82.7% when the irradiance of 40 mW/cm² and the dose of 40 J/cm² are applied under hyperoxic conditions. For reference, the graph of FIG. 25 also shows the average cell death percentage from the studies above where the irradiance of 2.5 mW/cm² and the dose of 2.5 J/cm² are applied under normoxic conditions, and where the irradiance of 40 mW/cm² and the dose of 40 J/cm² are applied under normroxic conditions.

TABLE 3
0.1% Riboflavin Concentration, UVA
Laser under Hyperoxic Conditions

| | Control A | Control B | A | B |
|---|---|---|---|---|
| Oxygen Conditions | normoxic | hyperoxic | hyperoxic | hyperoxic |
| Irradiance (mW/cm²) | — | — | 2.5 | 40 |
| Dose (J/cm²) | — | — | 2.5 | 40 |
| CFU - Replicate 1 | 41 | 45 | 12 | 4 |
| CFU - Replicate 2 | 35 | 53 | 52 | 9 |
| CFU - Replicate 3 | 46 | 54 | 34 | 14 |

In a further study employing the example procedure 2220, a riboflavin concentration of 0.22% was applied and photoactivated with a UVA laser at an irradiance of 2.5 mW/cm² and a dose of 2.5 J/cm² (applied for 16 minutes, 40 seconds) under hyperoxic conditions. Additionally, a riboflavin concentration of 0.22% was applied and alternatively photoactivated with a UVA laser at an irradiance of 40 mW/cm² and a dose of 40 J/cm² (applied for 16 minutes, 40 seconds) under hyperoxic conditions. TABLE 4 shows the results of this study against a control (growth of Pseudomonas aeruginosa under normoxic conditions without application of riboflavin and UVA light) for three replicates (n=3). FIG. 26 illustrates a graph indicating an average cell death percentage of 13.9% when the irradiance of 2.5 mW/cm² and the dose of 2.5 J/cm² are applied under hyperoxic conditions, and an average cell death percentage of 76.9% when the irradiance of 40 mW/cm² and the dose of 40 J/cm² are applied under hyperoxic conditions. For reference, the graph of FIG. 26 also shows the average cell death from the studies above where the riboflavin concentrations of 0.1% and 0.5% are each applied and photoactivated with (i) the irradiance of 2.5 mW/cm² and the dose of 2.5 J/cm² under normoxic conditions, and (ii) the irradiance of 40 mW/cm² and the dose of 40 J/cm² under normoxic conditions, and where the riboflavin concentration of 0.1% is applied and photoactivated with (iii) the irradiance of 2.5 mW/cm² and the dose of 2.5 J/cm² under hyperoxic conditions, and (iv) the irradiance of 40 mW/cm² and the dose of 40 J/cm² under hyperoxic conditions.

TABLE 4
0.22% Riboflavin Concentration, UVA
Laser under Hyperoxic Conditions

| | Control | A | B |
|---|---|---|---|
| Oxygen Conditions | normoxic | hyperoxic | hyperoxic |
| Irradiance (mW/cm²) | — | 2.5 | 40 |
| Dose (J/cm²) | — | 2.5 | 40 |
| CFU - Replicate 1 | 197 | 191 | 33 |
| CFU - Replicate 2 | 213 | 218 | 56 |
| CFU - Replicate 3 | 317 | 187 | 83 |

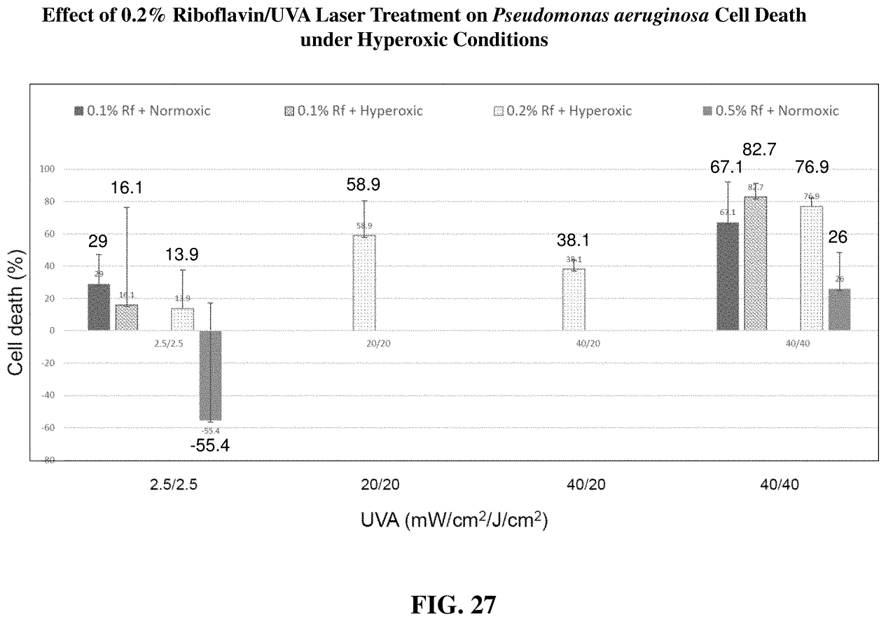

In yet a further study employing the example procedure 2220, a riboflavin concentration of 0.2% was applied and photoactivated with a UVA laser at an irradiance of 20 mW/cm² and a dose of 20 J/cm² (applied for 16 minutes, 40 seconds) under hyperoxic conditions. Additionally, a riboflavin concentration of 0.2% was applied and alternatively photoactivated with a UVA laser at an irradiance of 40 mW/cm² and a dose of 20 J/cm² (applied for 8 minutes, 20 seconds) under hyperoxic conditions. TABLE 5 shows the results of this study against controls (growth of Pseudomonas aeruginosa under normoxic conditions without application of riboflavin and UVA light) for three replicates (n=3). FIG. 27 illustrates a graph indicating an average cell death percentage of 58.9% when the irradiance of 20 mW/cm² and the dose of 20 J/cm² are applied under hyperoxic conditions, and an average cell death percentage of 38.1% when the irradiance of 40 mW/cm² and the dose of 20 J/cm² are applied under hyperoxic conditions. For reference, the graph of FIG. 27 also shows the average cell death from the studies above where the riboflavin concentrations of 0.1% and 0.5% are each applied and photoactivated with (i) the irradiance of 2.5 mW/cm² and the dose of 2.5 J/cm² under normoxic conditions, and (ii) the irradiance of 40 mW/cm² and the dose of 40 J/cm² under normoxic conditions, and where the riboflavin concentrations of 0.1% and 0.22% are each applied with (iii) the irradiance of 2.5 mW/cm² and the dose of 2.5 J/cm² under hyperoxic conditions, and (iv) the irradiance of 40 mW/cm² and the dose of 40 J/cm² under hyperoxic conditions.

TABLE 5
0.2% Riboflavin Concentration, UVA
Laser under Hyperoxic Conditions

| | Control A | Control B | A | B |
|---|---|---|---|---|
| Oxygen Conditions | normoxic | normoxic | hyperoxic | hyperoxic |
| Irradiance (mW/cm²) | — | — | 2.5 | 40 |
| Dose (J/cm²) | — | — | 2.5 | 20 |
| CFU - Replicate 1 | 139 | 81 | 114 | 77 |
| CFU - Replicate 2 | 137 | 66 | 140 | 78 |
| CFU - Replicate 3 | 166 | 28 | 101 | 63 |

According to an example procedure 2230 shown in FIG. 23C, a dilution factor is determined for Pseudomonas aeruginosa bacteria in step 2230a, and the Pseudomonas aeruginosa is loaded into wells in cell culture plate (e.g., a 96-well plate) in step 2230b. According to particular treatment parameters, a concentration of riboflavin in BBS is applied to the wells in step 2230c, and ultraviolet light is applied with an LED light source at a specified irradiance and dose to the wells under normoxic conditions in step 2230d. For example, the LED light source may emit UVA light at a size of approximately 6.5 mm. A petri dish is inoculated with the mixture from the wells in step 2230e, and the petri dish is incubated (e.g., overnight) in step 2230f. After incubation, the effect of the treatment parameters is evaluated by counting CFU in step 2230g.

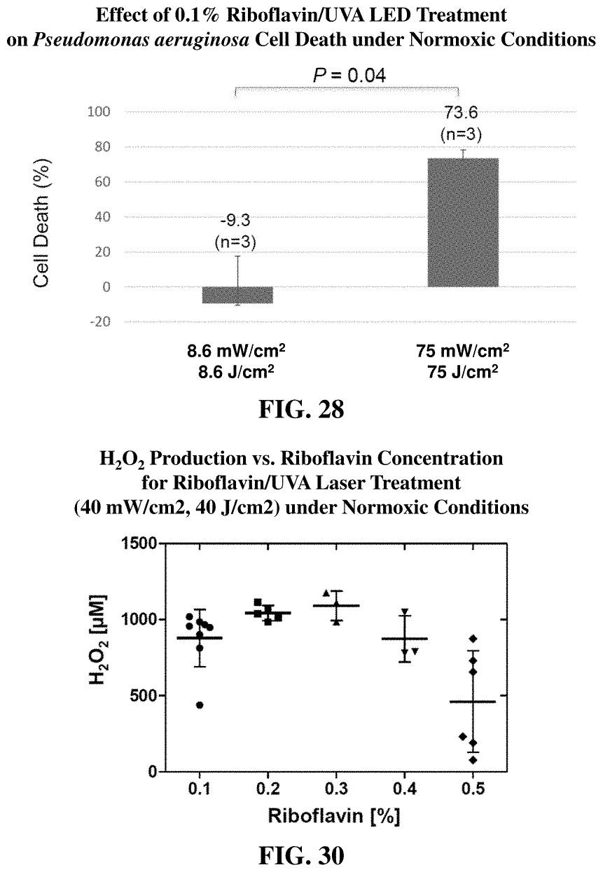

In a study employing the example procedure 2230, a riboflavin concentration of 0.1% was applied and photoactivated with UVA LED light at an irradiance of 8.6 mW/cm² and a dose of 8.6 J/cm² (applied for 16 minutes, 40 seconds) under normoxic conditions. Additionally, a riboflavin concentration of 0.1% was applied and alternatively photoactivated with UVA LED light at an irradiance of 75 mW/cm² and a dose of 75 J/cm² (applied for 16 minutes, 40 seconds) under normoxic conditions. TABLE 6 shows the results of this study against a control (growth of Pseudomonas aeruginosa under normoxic conditions without application of riboflavin and UVA light) for three replicates (n=3). FIG. 28 illustrates a graph indicating an average cell death percentage of -9.3% when the irradiance of 8.6 mW/cm² and the dose of 8.6 J/cm² are applied, and an average cell death percentage of 73.6% when the irradiance of 75 mW/cm² and the dose of 75 J/cm² are applied.

TABLE 6
0.1% Riboflavin Concentration, UVA
LED under Normoxic Conditions

| | Control | A | B |
|---|---|---|---|
| Irradiance (mW/cm²) | — | 8.6 | 75 |
| Dose (J/cm²) | — | 8.6 | 75 |
| CFU - Replicate 1 | 116 | 94 | 29 |
| CFU - Replicate 2 | 110 | 124 | 35 |
| CFU - Replicate 3 | 116 | 156 | 26 |

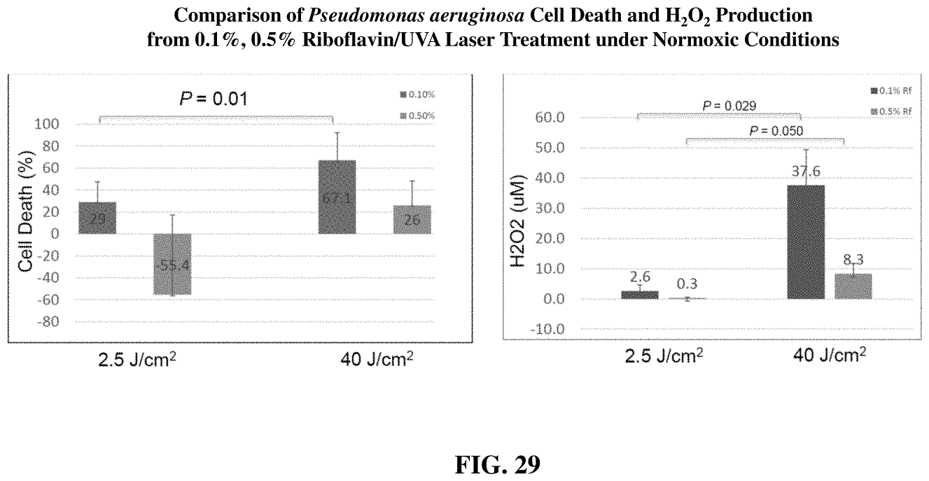

As described above, the reaction between riboflavin and UV light produces H₂O₂ and reactive oxygen species (ROS) (i.e., superoxide, singlet oxygen, and hydroxyl radical (OH)). FIG. 29 illustrates graphs comparing average cell death percentage for Pseudomonas aeruginosa and production of H₂O₂ (μM) from the studies above where the riboflavin concentrations of 0.1% and 0.5% are each applied with (i) an irradiance of 2.5 mW/cm² and a dose of 2.5 J/cm² under normoxic conditions, and (ii) an irradiance of 40 mW/cm² and a dose of 40 J/cm² under normoxic conditions. The graph of FIG. 29 shows that the production of H₂O₂ results in greater cell death. Meanwhile, FIG. 30 illustrates a graph showing production of H₂O₂ (μM) vs. riboflavin concentration applied and photoactivated with a UVA laser at an irradiance of 40 mW/cm² and a dose of 40 J/cm² under normoxic conditions. The graph of FIG. 30 indicates a range of concentrations of riboflavin (e.g., approximately between 0.2% and 0.4%) that can achieve greater production of H₂O₂ and greater cell death.

During the studies, the present inventors determined that supplemental oxygen does not create additional cross-linking effects when riboflavin is photoactivated with a scanning laser, but does create additional bacterial death. This is likely because the excess O₂ drives elevated H₂O₂ levels in situ, which is not useful for cross-linking but is useful for antimicrobial effect.

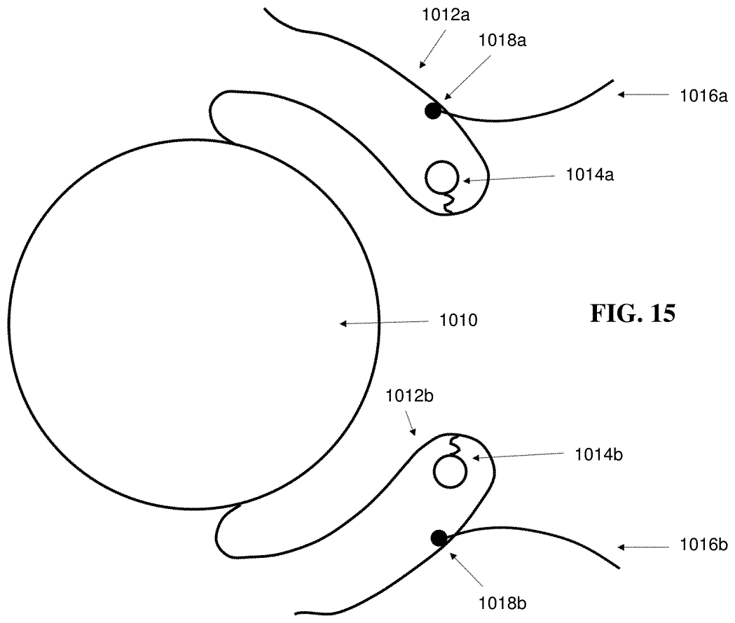

Illustrating anatomical features, FIG. 15 shows an eye 1010, an upper eyelid 1012a, a lower eyelid 1012b, Meibomian glands 1014a, b on the respective eyelids 1012a, b, and eyelashes 1016a, b extending from the respective eyelids 1012a, b with corresponding follicles 1018a, b. According to aspects of the present disclosure, treatments can reduce the burden of demodex mites and associated infection by bacillus oleronius in the Meibomian glands 1014a, b and/or eyelash follicles 1018a, b. Such treatments address the symptoms and root cause of blepharitis and restore Meibomian gland function. Such treatments may be applied once or repeatedly, alone or in combination with topical medications. Such treatments may reduce time to efficacy relative to treatments that rely solely on topical medications.

Example treatments for blepharitis may employ PDT. Although PDT treatments for keratitis may be specifically directed to corneal tissue as described above, aspects of the present disclosure also provide drug formulations and/or illumination systems that are optimized to sterilize other anatomical features via PDT, including but not limited to anatomical features experiencing infection associated with blepharitis. For instance, example drug formulations can be optimized to reduce the required amount of time that the infected regions are exposed to the photosensitizing agent (also known as soak time) before activating illumination is delivered. Example drug formulations can be optimized to maximize the antimicrobial effect. Example illumination systems can be optimized to localize the sterilization effects to the Meibomian glands 1014a, b and/or eyelash follicles 1018a, b to avoid unnecessary irritation of the surrounding tissue.

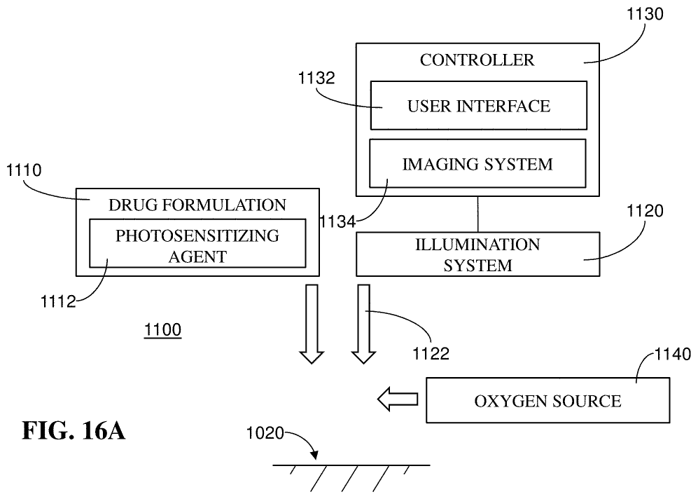

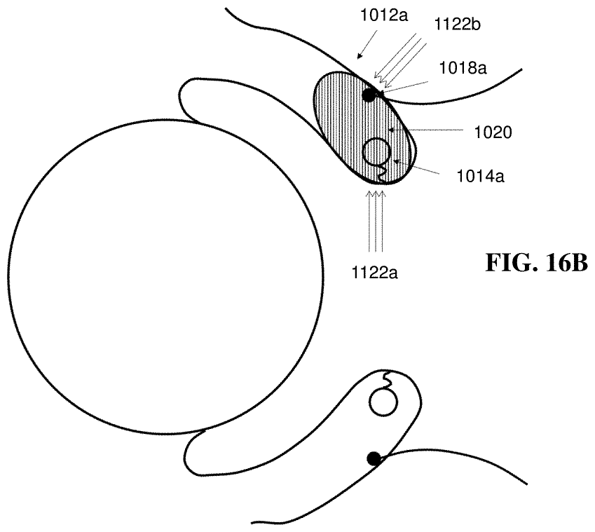

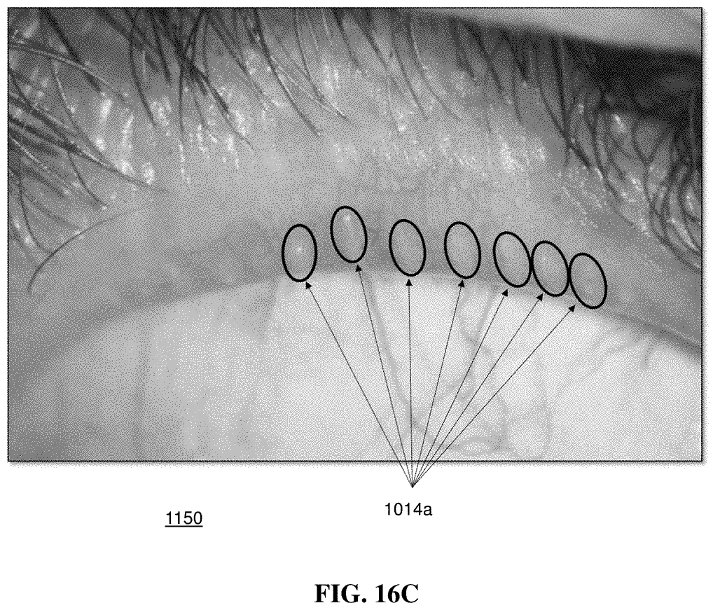

FIG. 16A illustrates an example system 1100 for applying PDT. Aspects of the system 1100 may be similar to the systems 100a, b described above. Correspondingly, FIG. 16B illustrates an example application of PDT as a treatment for blepharitis. In an implementation of the system 1100, a drug formulation 1110 including a photosensitizing agent 1112 is applied to a treatment area 1020 (e.g., as eye drops). The treatment area 1020, for instance, may include an area of the upper eyelid 1012a where blepharitis may occur. The photosensitizing agent 1112 may be riboflavin, 5-aminolevulinic acid (5-ALA), or the like.

Once the photosensitizing agent 1112 has permeated the treatment area 1020, an illumination system 1120 is operated to deliver illumination (radiation) 1122 that activates the photosensitizing agent 1112. The interaction of the photosensitizing agent 1112 and the illumination 1122 generates cytotoxic chemical species, such as reactive oxygen species (e.g., superoxide, singlet oxygen, and hydroxyl radical (OH)). Because these chemical species are highly toxic, activating a photosensitizing agent applied to the treatment area 1020 produces a sterilizing effect to treat blepharitis.

The illumination 1122 has a wavelength that matches an absorption peak of the photosensitizing agent 1112. If the photosensitizing agent 1112 is riboflavin, for example, the illumination 1122 may be ultraviolet light A (UVA) with a wavelength between approximately 350 nm and approximately 390 nm. On the other hand, if the photosensitizing agent 1112 is 5-ALA, the illumination 1122 may have a wavelength of approximately 600 nm. Because the skin surrounding the Meibomian glands 1014a and eyelash follicles 1018a may have a strong tendency to scatter light, however, it may be advantageous to employ longer wavelengths to improve depth of effect for the illumination 1122. As such, it may be advantageous to employ 5-ALA, instead of riboflavin, as the photosensitizing agent 1112. Alternatively, the photosensitizing agent 1112 may employ other compounds with absorption peaks in the near infrared (NIR) range which may provide further advantages.

The illumination system 1120 may employ a light emitting diode (LED) or a laser source to deliver the illumination 1122. Because laser beams can have diameters of approximately 50 μm to approximately 2 mm, use of a laser source for the illumination 1122 can allow more precise targeting of individual Meibomian glands 1014a and/or eyelash follicles 1018a. Such targeting can limit the generation of reactive oxygen species to areas of probable infection. Thus, as shown in FIG. 16B, the illumination 1122 can provide targeted activation 1122a of the photosensitizing agent 1112 at the Meibomian glands 1014a and/or targeted activation 1122b of the photosensitizing agent 1112 at the eyelash follicles 1018a (although the photosensitizing agent 1112 may be applied to the broader treatment area 1020). In some implementations, the laser beam can be sequentially scanned from gland to gland or from follicle to follicle. The laser beam can transition from one zone (individual gland or follicle) to another after each zone is completely treated. Alternatively, the laser beam can transition between zones in a repetitive pattern, where each zone receives multiple fractionated doses of the illumination 1122 and the time between doses allows oxygen at the zone to be replenished for further activation of the photoactivating agent 1112 by subsequent doses and further generation of reactive oxygen species. In alternative implementations, the illumination 1122 may be applied with a broader LED or laser pattern to a larger (less targeted) region of the eyelid 1012a, particularly if bacterial infection has spread beyond the Meibomian glands 1014a and eyelash follicles 1018a.

The system 1100 may also include a controller 1130 to control certain treatment parameters. For instance, the controller 1130 can be coupled to the illumination system 1120 to control parameters relating to the illumination 1122, such as instantaneous power, average irradiance, total dose, and pulsing characteristics, any and all of which can be varied spatially according infected areas of the treatment area 1020. Additionally, oxygen from an oxygen source 1140 may be delivered to the treatment area 1020 to determine a level of ambient oxygen for the treatment. The generation of the reactive oxygen species and the associated sterilizing effect can be controlled with different combinations of drug concentration, ambient oxygen level, light source irradiance, and/or treatment time.

The controller 1130 may also include a user interface 1132. The user interface 1132 can receive input from an operator to control the treatment parameters implemented by the controller 1130. Such input can be received, for instance, via a keyboard, computer mouse, touchscreen, stylus, dials, buttons, or the like. The user interface 1132 can also provide the operator with treatment information. Such treatment information can be provided, for instance, visually and/or audibly via a display, illuminated indicators, speakers, or the like.

Additionally, the controller 1130 may also include an imaging system 1134 with a camera that can provide images of the treatment area 1020. In particular, the images from the imaging system 1134 can be employed by machine vision algorithms to detect, track, and deliver the illumination 1122 specifically to the Meibomian glands 1014a and/or eyelash follicles 1018a. The controller 1130 may detect Meibomian glands 1014a and eyelash follicles 1018a automatically based on characteristic features of these anatomical structures. Alternatively, prior to treatment, an operator may receive the images of the treatment area 1020 and provide input via the user interface 1132 to identify the zones to be targeted with the illumination 1122 by the controller 1130. FIG. 16C illustrates an example image 1150 where Meibomian glands 1014a for treatment for blepharitis can be detected automatically by machine vision algorithms or identified by an operator's input into the user interface 1132. Similar detection/identification can be employed for eyelash follicles 1018a. The controller 1130 can ensure that the illumination 1122 is limited to treatment areas associated with the Meibomian glands or eyelash follicles.

To apply PDT, a holding device may be employed to evert and hold the eyelid 1012a in a position that permits direct delivery of the illumination 1122. This holding device may include a clip that fixes to a mechanical mount. Additionally, a light shield may employed to protect the corneal surface or non-targeted tissues (where treatment is not needed) from unwanted exposure to the illumination 1122. For example, the light shield may include an opaque contact lens that is positioned over the eye 1010.

In addition to PDT, other treatments for blepharitis may involve cryotherapy, radiofrequency (RF) therapy, direct thermal therapy, and/or ultrasound therapy to achieve desired sterilization effects. Advantageously, the sterilization effects for these other treatments may also be localized to the Meibomian glands 1014a, b and/or eyelash follicles 1018a, b to avoid unnecessary irritation of the surrounding tissue.

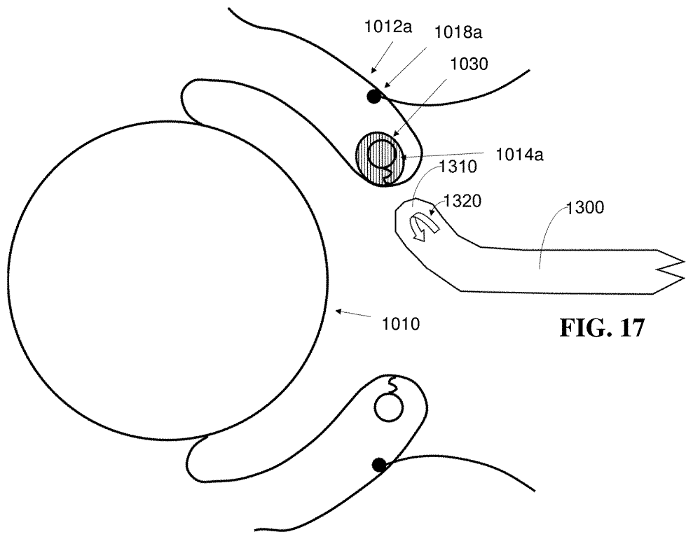

FIG. 17 illustrates an example application of cryotherapy as a treatment for blepharitis. The cryotherapy involves the localized application of temperatures that are sufficiently low to kill demodex mites. As shown in FIG. 17, an example cryotherapy device 1300 includes an active cryogenic tip 1310 (e.g., implemented on a handpiece). The cryogenic tip 1310 maintains a low temperature, for example between approximately −140° C. and approximately −90° C., and is placed into direct physical contact with a treatment area 1030 at the eyelid 1012a. To achieve low temperatures at the cryogenic tip 1310, a cryogenic fluid 1320, such as liquid nitrogen, may circulate internally within the cryotherapy device 1300. Any outgassing of ultracold materials particularly onto the eye 1010 may otherwise cause unwanted damage. The cryogenic tip 1310 may be sized so that the cryotherapy can be applied to a small number of Meibomian glands 1014a or eyelash follicles 1018a at a time. In an alternative implementation, instead of a cryogenic tip (e.g., on a handpiece), the cryotherapy device 1300 may include a cryogenic pad that is more broadly sized to treat the entire upper eyelid or lower eyelid 1018b at once. Such a cryogenic pad may have a “clamshell” configuration that can fold over or pinch the eyelid, where the cryotherapy can be applied via surface(s) in an interior portion while the eye 1010 remains protected from cryogenic conditions on the exterior.

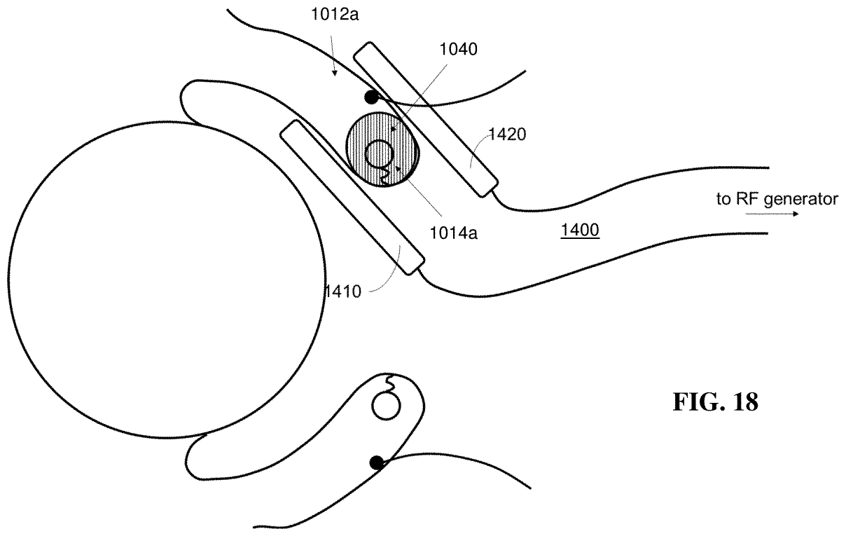

FIG. 18 illustrates an example application of RF therapy as a treatment for blepharitis. The RF therapy involves the localized application of temperatures that are sufficiently high to kill demodex mites and associated bacteria. For example, demodex mites cannot survive in temperatures greater than approximately 56° C. As shown in FIG. 18, an RF energy system 1400 includes an RF emitter pad 1410 and an RF ground pad 1420 coupled conductively to an RF generator. The RF emitter pad 1410 is placed on one side (e.g., inner surface) of the eyelid 1012a, while the RF ground pad 1420 is placed on the opposing side (e.g., outer surface) of the eyelid 1012a. RF energy is generated at the RF emitter pad 1410, while the RF ground pad 1420 acts as a sink for the RF energy and ensures that the RF field remains confined to the treatment area 1040. The RF energy passing through the eyelid 1012a elevates temperatures in the treatment area 1040. Absorption of RF energy in the tissue of the eyelid 1012a, particularly the lipid-rich Meibomian glands 1014a results in localized temperature increase. Given the thin nature of the eyelid 1012a, frequencies on the order of tens or hundreds of gigahertz (GHz) may be effective to achieve desired energy delivery. Alternatively, the RF frequency and RF pulsing parameters may be selected based on the RF absorption profile of lipids found in the Meibomian glands 1014a.

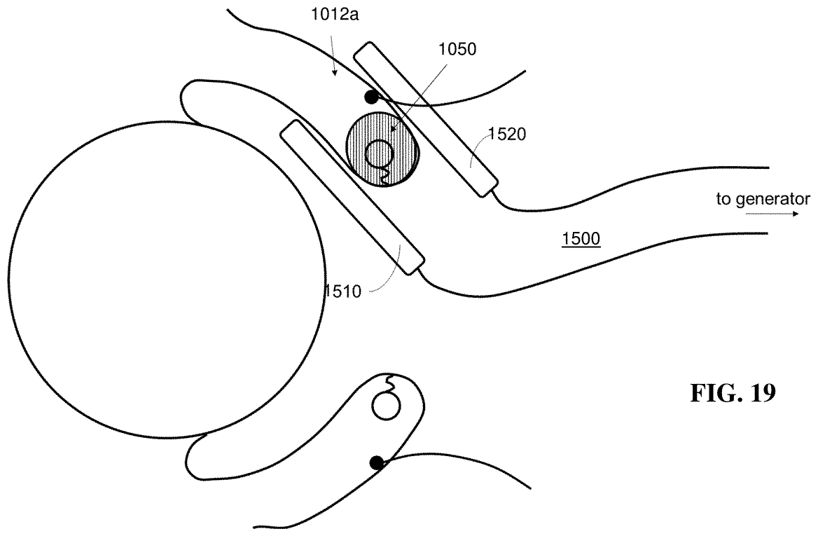

FIG. 19 illustrates an example application of direct thermal therapy via conductive heating elements as a treatment for blepharitis. As shown in FIG. 19, a direct thermal therapy system 1500 includes a first conductive heating element 1510 and a second conductive heating element 1520 coupled conductively to a generator. The first conductive heating element 1510 is placed on one side (e.g., inner surface) of the eyelid 1012a, while the second conductive heating element 1520 is placed on the opposing side (e.g., outer surface) of the eyelid 1012a. The conductive heating elements 1510, 1520 generate localized temperatures at a treatment area 1050 that are sufficiently high to kill demodex mites and associated bacteria.

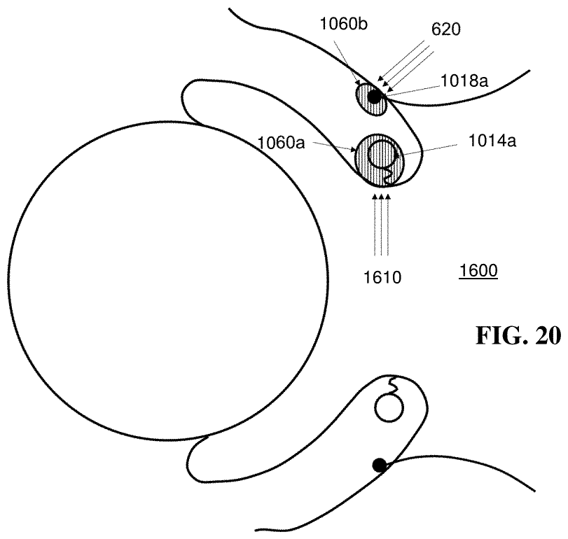

FIG. 20 illustrates an example application of direct thermal therapy via laser irradiation as a treatment for blepharitis. As shown in FIG. 20, a direct thermal therapy system 1600 directs targeted laser energy 1610 to a first treatment area 1060a corresponding to the Meibomian glands 1014a and directs targeted laser energy 1620 to a second treatment area 1060b corresponding to the eyelash follicles 1018a. The laser energy 1610, 1620 generate localized temperatures at the treatment areas 1060a, b that are sufficiently high to kill demodex mites and associated bacteria.

The direct thermal therapy system 1600 may include components similar to the illumination system 1120 and controller 1130 of the PDT system 1100 described above. As such, the laser energy 1610, 1620 can be precisely targeted to the treatment areas 1060a, b. For instance, beam focusing may be employed to apply the laser energy according to spot sizes of approximately 10 μm to approximately 200 μm. Additionally, the laser energy 1610, 1620 may be applied in pulses with nanosecond or picosecond durations to generate the desired energy density in the treatment areas 1060a, b. A sequence of pulses may be applied over a defined duration to allow the elevated temperature to be maintained for a sufficient duration to ensure death of demodex mites. Moreover, the direct thermal therapy system 1600 may employ a controller that can employ imaging and machine vision algorithms automatically to detect and track treatment areas. In alternative implementations, the controller can receive operator's input to identify the treatment areas.

In contrast to the PDT, however, the wavelengths for the laser energy 1610, 1620 correspond the optical absorption spectrum for the tissue in the treatment areas 1060a, b. For instance, the wavelength for the laser energy 1610 may correspond with the optical absorption spectrum of lipids in the Meibomian glands 1014a.

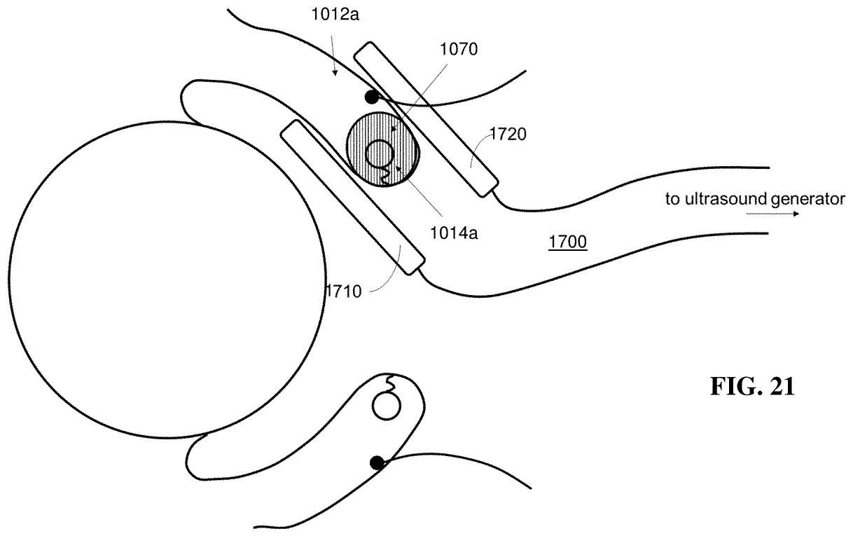

FIG. 21 illustrates an example application of ultrasound therapy as a treatment for blepharitis. The ultrasound therapy involves the localized application of shock waves that are sufficient to kill demodex mites. As shown in FIG. 21, an ultrasound therapy system 1700 includes an ultrasound emitter pad 1710 coupled to an ultrasound generator and an ultrasound sink pad 1720. The ultrasound emitter pad 1710 is placed on one side (e.g., inner surface) of the eyelid 1012a, while the ultrasound sink pad 1720 is placed on the opposing side (e.g., outer surface) of the eyelid 1012a. Given the thin nature of the eyelid, the ultrasound sink 1720 is employed to allow creation of a partial standing wave within the eyelid 1012a. High-frequency ultrasound waves are generated at the ultrasound emitter pad 1710. An impedance-matching gel may be applied on both sides of the eyelid 1012a to promote efficient energy transfer from the ultrasound emitter pad 1710. When exposed to the high-frequency ultrasound waves in surrounding tissue, demodex mites experience a mechanical shock and are damaged or destroyed due to the mismatch between their acoustic impedance and the surrounding tissue.

The embodiments described herein may employ controllers and other devices for processing information and controlling aspects of the example systems. For example, the example treatment systems 100a, b shown in FIGS. 1, 4 include the controller 130 and the system 1100 shown in FIG. 16A includes the controller 1130. Generally, the controller includes one or more processors. The processor(s) of a controller or other devices may be implemented as a combination of hardware and software elements. The hardware elements may include combinations of operatively coupled hardware components, including microprocessors, memory, signal filters, electronic/electric chip/circuit, etc. The processors may be configured to perform operations specified by the software elements, e.g., computer-executable code stored on computer readable medium. The processors may be implemented in any device, system, or subsystem to provide functionality and operation according to the present disclosure. The processors may be implemented in any number of physical devices/machines. Indeed, parts of the processing of the example embodiments can be distributed over any combination of processors for better performance, reliability, cost, etc.

The physical devices/machines can be implemented by the preparation of integrated circuits or by interconnecting an appropriate network of conventional component circuits, as is appreciated by those skilled in the electrical art(s). The physical devices/machines, for example, may include field programmable gate arrays (FPGA's), application-specific integrated circuits (ASIC's), digital signal processors (DSP's), etc.

Appropriate software can be readily prepared by programmers of ordinary skill based on the teachings of the example embodiments, as is appreciated by those skilled in the software arts. Thus, the example embodiments are not limited to any specific combination of hardware circuitry and/or software. Stored on one computer readable medium or a combination of computer readable media, the computing systems may include software or instructions executable by one or more processors for controlling the devices and subsystems of the example embodiments, for driving the devices and subsystems of the example embodiments, for enabling the devices and subsystems of the example embodiments to interact with a human user (user interfaces, displays, controls), etc. Such software can include, but is not limited to, device drivers, operating systems, development tools, applications software, etc. A computer readable medium further can include the computer program product(s) including executable instructions for performing all or a portion of the processing performed by the example embodiments. Computer program products employed by the example embodiments can include any suitable interpretable or executable code mechanism, including but not limited to complete executable programs, interpretable programs, scripts, dynamic link libraries (DLLs), applets, etc. The processors may include, or be otherwise combined with, computer-readable media. Some forms of computer-readable media may include, for example, a hard disk, any other suitable magnetic medium, any suitable optical medium, RAM, PROM, EPROM, flash memory, any other suitable memory chip or cartridge, any other suitable non-volatile memory, a carrier wave, or any other suitable medium from which a computer can read.

The controllers and other devices may also include databases for storing data. Such databases may be stored on the computer readable media described above and may organize the data according to any appropriate approach. For example, the data may be stored in relational databases, navigational databases, flat files, lookup tables, etc.

While aspects of the present disclosure have been described with reference to one or more particular embodiments, those skilled in the art will recognize that many changes may be made thereto without departing from the spirit and scope of the present disclosure. Each of these embodiments and obvious variations thereof is contemplated as falling within the spirit and scope of the present disclosure. It is also contemplated that additional embodiments according to aspects of the present disclosure may combine any number of features from any of the embodiments described herein.

What is claimed is:

1. A method for antimicrobial treatment, comprising:
detecting an ulcerative region on a cornea;
applying a photosensitizing agent to the cornea; and
delivering, with an illumination system, an illumination that activates the photosensitizing agent applied to the ulcerative region according to a set of parameters for treating the ulcerative region, wherein the illumination is restricted to the ulcerative region, and activation of the photosensitizing agent in the ulcerative region generates an antimicrobial effect.

2. The method of claim 1, further comprising:
identifying at least one additional treatment zone on the cornea outside the ulcerative region; and
delivering, with the illumination system, additional illumination that activates the photosensitizing agent applied to the at least one additional treatment zone according to a set of parameters for treating the at least one additional treatment zone, wherein the set of parameters for treating the at least one additional treatment zone is different from the set of parameters for treating the ulcerative region, and the additional illumination is restricted to the at least one additional treatment zone.

3. The method of claim 2, wherein the at least one additional treatment zone is defined by a border of the ulcerative region.

4. The method of claim 2, wherein the at least one additional treatment zone is defined by a peripheral region beyond a border of the ulcerative region.

5. The method of claim 2, wherein activation of the photosensitizing agent in the at least one additional treatment zone generates cross-linking activity.

6. The method of claim 1, wherein the antimicrobial effect is associated with at least one of:
(i) oxygen depletion; (ii) generation of singlet oxygen; or (iii) generation of hydrogen peroxide.

7. The method of claim 6, further comprising determining the set of parameters for treating the ulcerative region to increase at least one of: (i) the oxygen depletion; (ii) the generation of singlet oxygen; or (iii) the generation of hydrogen peroxide.

8. The method of claim 1, wherein the set of parameters for treating the ulcerative region includes at least one of: a concentration of the photosensitizing agent, a level of ambient oxygen applied to the cornea, an irradiance of the illumination, or a time for applying the photosensitizing agent to the cornea or delivering the illumination.

9. The method of claim 1, further comprising applying, from an oxygen source, a concentration of ambient oxygen to the cornea.

10. The method of claim 1, wherein detecting an ulcerative region on a cornea includes:
providing an image of the cornea on a display of a user interface;
receiving, via the user interface, input identifying a point within the ulcerative region as shown in the image; and
processing the image to identify a border of the ulcerative region.

11. The method of claim 1, wherein detecting an ulcerative region on a cornea includes:
providing an image of the cornea on a display of a user interface; and
receiving, via the user interface, input identifying a border of the ulcerative region.

12. The method of claim 1, further comprising:
tracking a change in a location of the ulcerative region relative to the illumination system,
wherein the illumination is delivered to the ulcerative region based on the change in the location.

13. The method of claim 1, wherein the photosensitizing agent is riboflavin and the illumination is ultraviolet (UV) light.

14. An antimicrobial treatment system comprising:
an illumination system configured to deliver illumination that activates a photosensitizing agent applied to a cornea; and
a controller configured to control the illumination system, the controller including one or more processors and one or more computer readable media, the one or more processors configured to execute instructions from the computer readable media to cause the controller to:
detect an ulcerative region on a cornea; and
cause the illumination system to deliver the illumination to activate the photosensitizing agent applied to the ulcerative region according to a set of parameters for treating the ulcerative region, wherein the illumination is restricted to the ulcerative region, and activation of the photosensitizing agent in the ulcerative region generates an antimicrobial effect.

15. The antimicrobial treatment system of claim 14, wherein the one or more processors are configured to execute instructions from the computer readable media to further cause the controller to:
identify at least one additional treatment zone on the cornea outside the ulcerative region; and
cause the illumination system to deliver additional illumination to activate the photosensitizing agent applied the at least one additional treatment zone according to a set of parameters for treating the at least one additional treatment zone, wherein the set of parameters for treating the at least one additional treatment zone is different from the set of parameters for treating the ulcerative region, and the additional illumination is restricted to the at least one additional treatment zone.

16. The antimicrobial treatment system of claim 15, wherein the at least one additional treatment zone is defined by a border of the ulcerative region.

17. The antimicrobial treatment system of claim 15, wherein the at least one additional treatment zone is defined by a peripheral region beyond a border of the ulcerative region.

18. The antimicrobial treatment system of claim 15, wherein activation of the photosensitizing agent in the at least one additional treatment zone generates cross-linking activity.

19. The antimicrobial treatment system of claim 14, wherein the antimicrobial effect is associated with at least one of: (i) oxygen depletion; (ii) generation of singlet oxygen; or (iii) generation of hydrogen peroxide.

20. The antimicrobial treatment system of claim 19, wherein the one or more processors are configured to execute instructions from the computer readable media to further cause the controller to determine the set of parameters for treating the ulcerative region to increase at least one of: (i) the oxygen depletion; (ii) the generation of singlet oxygen; or (iii) the generation of hydrogen peroxide.

21. The antimicrobial treatment system of claim 14, wherein the set of parameters for treating the ulcerative region includes at least one of: a concentration of the photosensitizing agent, a level of ambient oxygen applied to the cornea, an irradiance of the illumination, or a time for applying the photosensitizing agent to the cornea or delivering the illumination.

22. The antimicrobial treatment system of claim 14, further comprising an oxygen source configured to provide a concentration of ambient oxygen to the cornea.

23. The antimicrobial treatment system of claim 14, wherein the controller includes a user interface with a display, and the one or more processors are configured to execute instructions from the computer readable media to further cause the controller to:
provide an image of the cornea on the display of the user interface;
receive, via the user interface, input identifying a point within the ulcerative region as shown in the image; and
process the image to identify a border of the ulcerative region.

24. The antimicrobial treatment system of claim 14, wherein the controller includes a user interface with a display, and the one or more processors are configured to execute instructions from the computer readable media to further cause the controller to:
provide an image of the cornea on the display of the user interface; and
receive, via the user interface, input identifying a border of the ulcerative region.

25. The antimicrobial treatment system of claim 14, wherein the one or more processors are configured to execute instructions from the computer readable media to further cause the controller to:
track a change in a location of the ulcerative region relative to the illumination system,
wherein the illumination is delivered to the ulcerative region based on the change in the location.

26. The antimicrobial treatment system of claim 14, wherein the photosensitizing agent is riboflavin and the illumination is ultraviolet (UV) light.
